SEQUENCE LISTING (11 sequences)

SEQ ID NO: 1 - RNA, 21 nt, artificial sequence
  synthetically produced sequence
aagagccgcgagucguacgug

SEQ ID NO: 2 - RNA, 21 nt, artificial sequence
  synthetically produced sequence
aacgcaccuguggaugaggcc

SEQ ID NO: 3 - RNA, 21 nt, artificial sequence
  synthetically produced sequence
cgggcuucggggcugcucuuu

SEQ ID NO: 4 - RNA, 19 nt, artificial sequence
  synthetically produced sequence
augccuggaacaaccugga

SEQ ID NO: 5 - RNA, 18 nt, artificial sequence
  synthetically produced sequence
ccagguuguuccaggcau

SEQ ID NO: 6 - RNA, 19 nt, artificial sequence
  synthetically produced sequence
augccuggaacaaccugga

SEQ ID NO: 7 - RNA, 19 nt, artificial sequence
  synthetically produced sequence
uccagguuguuccaggcau

SEQ ID NO: 8 - RNA, 21 nt, artificial sequence
  synthetically produced sequence
aagagccgcgagucguacgug

SEQ ID NO: 9 - RNA, 21 nt, artificial sequence
  synthetically produced sequence
cgggcuucggggcugcucuuu

SEQ ID NO: 10 - DNA, 51 bp, artificial sequence
  synthetically produced sequence
caccgcttccgtctctatttatattcaagagatataaatagagacggaagc

SEQ ID NO: 11 - DNA, 51 bp, artificial sequence
  synthetically produced sequence
aaaagcttccgtctctatttatatctcttgaatataaatagagacggaagc

(19) United States
(12) Patent Application Publication
(10) Pub. No.: US 2014/0093494 A1
(43) Pub. Date: Apr. 3, 2014

(54) ALPHA SYNUCLEIN TOXICITY

(71) Applicants: University of Graz, Graz (AT); Katholieke Universiteit Leuven, Leuven (BE)

(72) Inventors: Veerle Baekelandt, Heverlee (BE); Sabrina Buettner, Graz (AT); Frank Madeo, Graz (AT); Joris Winderickx, Wilsele (BE)

(73) Assignees: University of Graz, Graz (AT); Katholieke Universiteit Leuven, Leuven (BE)

(21) Appl. No.: 13/972,683

(22) Filed: Aug. 21, 2013

Related U.S. Application Data
(63) Continuation of application No. 12/673,225, filed on Oct. 5, 2011, now abandoned.
(60) Provisional application No. 61/133,728, filed on Jul. 2, 2008.

(30) Foreign Application Priority Data
Aug. 14, 2007 (GB) 0715809.0

(51) Int. Cl. A61K 39/395; A61K 38/00; A61K 31/713

(52) U.S. Cl. CPC A61K 39/3955; A61K 31/713; A61K 38/005
USPC 424/133.1; 514/44 R; 424/158.1; 514/44 A; 514/1.1; 424/146.1

(57) ABSTRACT

It is demonstrated that alpha synculein toxicity such as α-synuclein mediated cell death, and alpha synuclein induced reactive oxygen species (ROS) in a cell requires proapoptotic endonuclease G and that the deletion of the endonuclease G or suppressing of the endonuclease G apoptotic pathway attenuates or counteracts such alpha synuclein toxicity. In view of these observations, compositions and methods for inhibition of α-synuclein toxicity are provided. The inhibiting α-synuclein toxicity can be used in methods for the treatment of synucleinopathies, such as Parkinsons disease (PD), dementia with Lewy bodies (DLB), pure autonomic failure (PAF), and multiple system atroypy (MSA) and the manufacture of medicaments for such treatment. In particular, pharmaceutical compositions containing inhibitors of endonuclease G, and their use in the treatment of synucleinopathies such as Parkinson's disease, dementia with Lew bodies, pure autonomic failure, and multiple system atrophy and the manufacture of medicaments for such treatment are presented. In addition, methods for the identification of compounds for attenuating the synuclein toxicity are also provided.

### RELATED APPLICATIONS

This application is a continuation of U.S. application Ser. No. 12/673,225 filed Oct. 5, 2010, which application claimed priority to PCT Application PCT/BE2008/00062 filed Aug. 7, 2008, which application claimed priority to U.S. Provisional Application 61/133,728 filed Jun. 30, 2008 and Great Britain Patent Application 0715809.0 filed Aug. 14, 2007.

### FIELD OF THE INVENTION

The present invention concerns compounds, compositions and methods for inhibiting α-synuclein toxicity. Such compounds, compositions can be used in methods of treatment of synucleinopathies, such as Parkinson's disease (PD), dementia with Lewy bodies (DLB), pure autonomic failure (PAF), and multiple system atrophy (MSA). Moreover the subject matter provided in this invention relates to a pharmaceutical compositions containing inhibitors of endonuclease G, and their use in the treatment of synucleinopathies, such as Parkinson's disease, dementia with Lewy bodies, pure autonomic failure, and multiple system atrophy and the use of endonuclease G antagonists that inhibit the expression or activity of endonuclease G for the manufacture of medicaments for such treatment. Another aspect of present invention is a method for the identification of compounds attenuating the synuclein toxicity, said method comprising evaluating the inhibitory action of said compound on the endonuclease G dependent apoptosis.

### BACKGROUND OF THE INVENTION

To study the biochemistry and pathogenicity of α-synuclein, several model systems have been developed ranging from flies and worms to transgenic mice. Studies with these models pointed to proteasomal dysfunction and oxidative stress pathways as important factors determining α-synuclein toxicity and implicated mitochondria as a possible site of action (Gosal et al., 2006; Moore et al., 2005). However, the downstream events or cell death executors required for α-synuclein mediated death remained elusive.

Most recently, the yeast Saccharomyces cerevisiae was added to the list of validated model systems for studies on α-synuclein. Reminiscent to data produced by other models, the yeast system showed α-synuclein to localize to the plasma membrane, to form thioflavin-S positive intracellular inclusions, to influence vesicular trafficking and endocytosis, and to inhibit phospholipase D (Dixon et al., 2005; Outeiro and Lindquist, 2003; Zabrocki et al., 2005).

In recent years, yeast has also been established as a model of apoptosis as it undergoes cell death accompanied by typical apoptotic markers (Madeo et al., 1997; Madeo et al., 1999) (Ludovico et al., 2001). Moreover, the basic molecular machinery executing apoptotic cell death seems to be conserved, as orthologues of caspases (Madeo et al., 2002), the apoptosis inducing factor (Wissing et al., 2004), and the serine protease OMI/HtrA2 (Fahrenkrog et al., 2004) have been described. In addition, yeast apoptotic death occurs in dependency of complex apoptotic scenarios such as mitochondrial fragmentation (Fannjiang et al., 2004), cytochrome C release (Ludovico et al., 2002), cytoskeletal perturbations (Gourlay et al., 2004) or ageing (Herker et al., 2004; Laun et al., 2001). Finally, yeast cells, and in particular chronological aged yeast cells, are currently used as a valuable model to study oxidative damage and molecular conserved ageing pathways of post-mitotic tissues in higher organisms (Longo, 1999) and recent evidence has shown that aged yeast cells die exhibiting an apoptotic phenotype (Fabrizio et al., 2004) (Herker et al., 2004; Laun et al., 2001).

In this study, we introduce an ageing yeast model for Parkinson's disease as we used chronological aged yeast as a model to mimic age-induced neurodegeneration. We demonstrate that indeed ageing is a trigger for apoptotic and necrotic cell death upon α-synuclein expression. Moreover, we use the unique possibility to manipulate mitochondrial function in yeast and demonstrate that abrogation of mitochondrial DNA (rho₀) not only delays synuclein facilitated death, but also efficiently suppresses ROS formation. Consistently, we identified mitochondrial endonuclease G, as a key executor of cell death induced by synuclein.

The study clearly demonstrates that α-synuclein toxicity such as α-synuclein mediated cell death and α-synuclein induced reactive oxygen species (ROS) in a cell requires the proapoptotic endonuclease G. Moreover the study demonstrates that the deletion of endonuclease G or the suppressing of the endonuclease G apoptotic pathway attenuates or counteracts such α-synuclein toxicity. Compounds that antagonise endonuclease G nuclease activity, compositions containing such compounds, and methods of use of such compounds have been provided by present invention for reducing or preventing α-synuclein toxicity. Furthermore methods of treatment of α-synuclein toxicity by inhibiting endonuclease G nuclease activity or the endonuclease G apoptotic pathway and the manufacture of medicaments for such treatment are an object of the present invention.

In the scope of the invention is a method for the identification of compounds, which attenuate the α-synuclein toxicity, said method comprising evaluating the inhibitory action of said compound on the endonuclease G dependent apoptosis. Such can comprise the monitoring of the survival of yeast cells overexpressing endonuclease G or a homolog thereof in absence or presence of said compound. The enhancement of the survival of the yeast cell in presence of said compound is indicative for an inhibitory action of the compound on endonuclease G dependent apoptosis. Furthermore the method can comprise comparing the evolution of apoptotic markers in yeast cells overexpressing endonuclease G or a homolog thereof in absence or presence of said compound. Furthermore the apoptotic marker in such method can be selected out of the group consisting of accumulation of ROS, DNA fragmentation, externalization of phosphadityl serine and membrane permeabilization. The endonuclease G homolog used can be the yeast endonuclease G homolog, Nuc1p. Furthermore the yeast cells can be exposed to an apoptotic trigger. In a particular embodiment the apoptotic trigger is a 0.4 mM peroxide or the exposure to metal ions, such as Fe3+ or Zn2+. Furthermore the method can comprise the monitoring of the inhibitory action of a compound on the endonuclease activity of endonuclease G or a homolog thereof. In a particular embodiment the inhibitory action of said compound on the endonuclease activity of endonuclease G is tested in an acid solubilisation endonuclease assay. In a particular embodiment the monitoring of the effect of a compound on the protein-protein interaction between an endonuclease G homolog and an interaction partner of endonuclease G in the apoptotic pathway. In yet another embodiment the interaction partner of endonuclease G is selected out of the group consisting of the proteins involved in the mitochondrial permeability transition pore complex (PTPC), the karyopherin Kap123 and the histone H2B. The interference of said molecule in the protein-protein interaction can for this method be investigated using a Fluorescence Resonance Energy Transfer (FRET) assay.

Provided are methods of treatment or amelioration of one or more symptoms of diseases and disorders associated with α-synuclein toxicity. Also provided are methods of treatment or amelioration of one or more symptoms of diseases and disorders associated with α-synuclein fibril formation. Such diseases and disorders include, but are not limited to, Parkinson's disease and Lewy body dementia. Other diseases and disorders include synucleinopathies, such as pure autonomic failure, and multiple system atrophy.

Use of any of the described compounds for the treatment or amelioration of one or more symptoms of diseases and disorders associated with α-synuclein toxicity or α-synuclein fibril formation is also contemplated. Furthermore, use of any of the described compounds for the manufacture of a medicament for the treatment of diseases and disorders associated with α-synuclein toxicity or α-synuclein fibril formation is also contemplated.

The present invention also provides a method of inhibiting or preventing α-synuclein toxicity such as oxidative stress induced by alpha-synuclein or necrosis induction by α-synuclein by administering a composition that comprises at least one endonuclease G inhibitor to a mammal or contacting such with a cell.

### ILLUSTRATIVE EMBODIMENTS OF THE INVENTION

### Summary of the Invention

The present invention is based on the surprising finding that the proapoptotic endonuclease G is required for α-synuclein mediated cell death. This finding indicated that the synuclein toxicity can be attenuated by intervening in the endonuclease G apoptotic pathway such that the endonuclease G catalyzed DNA degradation and the subsequent production of reactive oxygen species (ROS) is counteracted. Suppressing the endonuclease G activity indeed reduces the α-synuclein toxicity, α-synuclein induced cell oxidative stress, α-synuclein induction lesions or cell death. Such interventions have been proposed as a pharmaceutical treatment by the present invention.

Therefore, it is a first object of present invention to provide the use of compounds having an inhibitory action on endonuclease G dependent apoptosis in the manufacture of a medicine for the treatment of α-synuclein toxicity associated diseases or synucleinopathies, such as Parkinson's disease (PD), dementia with Lewy bodies (DLB), pure autonomic failure (PAF), or multiple system atrophy (MSA).

A first embodiment of this object is a compound having an inhibitory action on endonuclease G dependent apoptosis or that inhibits the expression and/or activity of endonuclease G for use in a treatment to cure or to prevent of α-synuclein toxicity associated diseases for instance to cure or to prevent synucleinopathies or such α-synuclein toxicity associated diseases of the group consisting of Parkinson's disease, dementia with Lewy bodies, pure autonomic failure and multiple system atrophy. Such compound having an inhibitory action on endonuclease G dependent apoptosis or inhibiting the expression and/or activity of endonuclease G can selected from the group consisting of a nucleotide, an antibody, a ribozyme, and a tetrameric peptide. To enhance cell entry such compound can be conjugated with a protein transduction domain.

The nucleotide to inhibit the expression and/or activity of endonuclease G can be an antisense DNA or RNA, siRNA, miRNA or an RNA aptamer. Other suitable reducing α-synuclein activity are the monoclonal antibodies specifically directed to endonuclease G or antigen-binding fragment thereof. Such antibody or antibody fragment can be humanized.

A second embodiment of this first object concerns the use of a compound having an inhibitory action on endonuclease G dependent apoptosis or inhibit the expression and/or activity of endonuclease G in the manufacture of a medicament for the treatment of α-synuclein toxicity associated diseases for instance such a synucleinopathy as Parkinson's disease, dementia with Lewy bodies, pure autonomic failure or multiple system atrophy. endonuclease G antagonists that are available or that can be produced with current state of the art technology are inhibiting nucleotides, antibodies, ribozymes or tetrameric peptides.

In a second object of the present invention to provide a method for the identification of compounds attenuating the synuclein toxicity, such as the necrosis induction by alpha synuclein, said method comprising evaluating the inhibitory action of said compound on the endonuclease G dependent apoptosis. In a first embodiment said method comprises the monitoring of the survival of yeast cells overexpressing endonuclease G or the yeast homolog of endonuclease G, Nuc1p, in absence or presence of said compound. Enhancement of the survival of the yeast cell in presence of said compound is indicative for an inhibitory action of the compound on endonuclease G dependent apoptosis. In a more preferred embodiment, the yeast cells overexpressing an endonuclease G or homolog thereof are exposed to an apoptotic trigger, such as a low concentration (for instance 0.4 mM) of peroxide or exposure to metal ions, for instance Fe³+(2-5 mM FeCl₃) or Zn²⁺ (8-16 mM ZnS0₄). Next to monitoring the survival of said yeast cells the inhibitory action of the compound can be investigated by comparing the evolution of apoptotic markers in said yeast cells incubated in presence. or absence of the compound. Suitable apoptotic markers are accumulation of ROS, DNA fragmentation, externalization of phosphadityl serine and membrane permeabilization.

In a second embodiment said method comprises the in vitro evaluation of the inhibitory action of a compound on the nuclease activity of endonuclease G. In a particular embodiment the inhibitory action of a compound on the nuclease activity of endonuclease G can be tested by comparing the activity of an isolated endonuclease G in an acid solubilisation endonuclease assay in presence or absence of such compound. Ikeda and Ozaki describe the isolation of endonuclease G (Ikeda and Ozaki, 1997), while Ikeda, S., Tanaka, T., Hasegawa, H., and Ozaki, K. discloses how to carry out the acid solubilisation endonuclease assay (Ikeda et al., 1996).

In a third embodiment the method of the present invention comprises the monitoring of the effect of a compound on the protein-protein interaction between an endonuclease G homolog and the interaction partners of endonuclease G in the apoptotic pathway. Within this pathway the proteins involved in the mitochondrial permeability transition pore complex (PTPC), the karyopherin Kap123 and the histone H2B are the main interaction partners of endonuclease G. The interference of a molecule in a protein-protein interaction can be investigated using a Fluorescence Resonance Energy Transfer (FRET) assay. The attenuating action on alpha synuclein toxicity or on the oxidative stress induced by alpha-synuclein or on the necrosis induction by alpha synuclein of molecules, which exhibit an inhibitory action on endonuclease G dependent apoptosis, can be subsequently investigated in yeast strain overexpressing α-synuclein or a variant thereof.

Small molecules, e.g. small organic molecules, and other drug candidates obtained, for example, from combinatorial and natural product libraries which attenuate the α-synuclein toxicity, can by the method of present invention be identification by evaluating the inhibitory action of said compound on the endonuclease G dependent apoptosis and by monitoring of the survival of yeast cells overexpressing endonuclease G or a homolog thereof of yeast cells overexpressing BNIP3 or a homolog thereof or cells in absence or presence of said compound. The use of such yeast cells overexpressing endonuclease G for identification of such small molecules that attenuate the α-synuclein toxicity or any such screenings system or screening apparatus comprising such yeast cells overexpressing endonuclease G is thus part of present invention.

### DEFINITIONS AND EXPLANATIONS

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, α-synuclein refers to one in a family of structurally related proteins that are prominently expressed in the central nervous system. Aggregated α-synuclein proteins form brain lesions that are hallmarks of some neurodegenerative diseases (synucleinopathies). The gene for α-synuclein, which is called SNCA, is on chromosome 4q21. Alpha-synuclein is a member of the synuclein family, which also includes beta- and gamma-synuclein. Synucleins are abundantly expressed in the brain and alpha- and beta-synuclein inhibit phospholipase D2 selectively. (Ueda, K.; et al. Proc. Nat. Acad. Sci. 90: 11282-11286, 1993) isolated an apparently full-length eDNA encoding a 140-amino acid protein within which 2 previously unreported amyloid sequences were encoded in tandem in the mouse hydrophobic domain. Campion, D.; et al. (Genomics 26: 254-257, 1995) cloned 3 alternatively spliced transcripts in lymphocytes derived from a normal subject, while Jakes, R. et al (FEBS Lett. 345: 27-32, 1994) identified two distinct synucleins from human brain and Beyer, K.; et al. (Neurogenetics 9: 15-23, 2008) identified and characterized a new alpha-synuclein isoform and its role in Lewy body diseases. These defined structures are hereby incorporated into the definition of α-synuclein.

As used herein “endonuclease G” or “EndoG” refers to a nuclear-encoded mitochondrial nuclease that has been reported to function in apoptosis, DNA recombination and cell proliferation. The protein encoded by this gene is a nuclear encoded endonuclease that is localized in the mitochondrion. The encoded protein is widely distributed among animals and cleaves DNA at GC tracts. This protein is capable of generating the RNA primers required by DNA polymerase gamma to initiate replication of mitochondrial DNA. (Cote, J. and Ruiz-Carrillo, A. (1993) Science 261, 765-769; Parrish, J. et al. (2001) Nature 412, 90-94.; Li, L. Y. et al. (2001) Nature 412, 95-99; Zhang, J. et al. (2003) Proc. Natl. Acad. Sci. USA 100, 15782-15787 and Huang, K. J. et al. (2006) Proc. Natl. Acad. Sci. USA 103, 8995-9000.) Homo sapiens endonuclease G, mRNA (cDNA clone MGC:4842 complete cds and its sequence) has for instance been described by Strausberg, R. L. et al. in Proc. Natl. Acad. Sci. U.S.A. 99 (26), 16899-16903 (2002) and the Homo sapiens endonuclease G (ENDOG), nuclear gene encoding mitochondrial protein, mRNA and its sequence has for instance been described by Varecha, M. et al. in Apoptosis 12 (7), 1155-1171 (2007).

As used herein “BNIP3” refers to a gene that is a member of the BCL2/adenovirus E1B 19 kd-interacting protein (BNIP) family. This gene contains a BH3 domain and a transmembrane domain, which have been associated with proapoptotic function. The dimeric mitochondrial protein encoded by this gene is known to induce apoptosis. The gene is located on the 10q26.3 chromosome. The sequence Homo sapiens BCL2/adenovirus E1B 19 kDa interacting protein 3 (BNIP3), nuclear gene encoding mitochondrial protein, mRNA has been also deposited as NM_{—}004052, 1535 bp, mRNA linear on 25 May 2008 and described by Ikeda, R., et al. Biochem. Biophys. Res. Commun. 370 (2), 220-224 (2008) and Azad, M. B et al. In Autophagy 4 (2), 195-204 (2008). BNIP3 is known to induce EndoG translocation and inhibition of BNIP3 expression significantly delayed EndoG translocation (Hou S T, MacManus J P. Int Rev Cytol. 2002; 221:93-148 and Zhengfeng Zhang et al; Stroke. 2007; 38:1606-1613).

The term synucleinopathies is used to name a group of neurodegenerative disorders characterized by fibrillary aggregates of alpha-synuclein protein in the cytoplasm of selective populations of neurons and glia. These disorders include Parkinson's disease (PO), dementia with Lewy bodies (DLB), pure autonomic failure (PAF), and multiple system atrophy (MSA). Clinically, they are characterized by a chronic and decline in motor, cognitive, behavioral, and autonomic functions, depending on the distribution of the lesions. Because of clinical overlap, differential diagnosis is sometimes very difficult.

Multiple system atrophy (MSA) is a sporadic neurodegenerative disorder that encompasses olivopontocerebellar atrophy (OPCA), striatonigral degeneration (SND) and Shy-Drager syndrome (SDS). The formation of alpha-synuclein aggregates is a critical event in the pathogenesis of multiple system atrophy (MSA). The histopathological hallmark is the formation of α-synuclein-positive glial cytoplasmic inclusions (GCis) in oligodendroglia. α-synuclein aggregation is also found in glial nuclear inclusions, neuronal cytoplasmic inclusions (NCis), neuronal nuclear inclusions (NNis) and dystrophic neuritis (Yoshida, Mari, Neuropathology, Volume 27, Number 5, October 2007, pp. 484-493(10)) and Ozawa T, 1: J Neurol Neurosurg Psychiatry. 2006 April; 77(4):464-7).

Parkinson's disease (PO) is the second most common age-associated neurodegenerative disease. Several observations suggested malfunctioning of the protein α-synuclein to be a toxic trigger of the neurodegenerative process during PO (Tofaris and Spillantini, 2005). In addition, three missense mutations (A30P, A53T and E46K) in the α-synuclein gene are linked to early-onset dominant familial PD. More recently, overexpression of wild-type α-synuclein due to gene duplication or triplication was found to be sufficient to cause a familial form of PO (Hardy et al., 2006). So far, studies using existing in vitro or in vivo models demonstrated that α-synuclein has roles in lipid and vesicle dynamics (Chandra et al., 2005; Larsen et al., 2006; Sidhu et al., 2004) but its exact function remains elusive.

Dementia with Lewy bodies is the second most frequent cause of hospitalization for dementia, after Alzheimer's disease. Current estimates are that about 60-to-75% of diagnosed dementias are of the Alzheimer's and mixed (Alzheimer's and vascular dementia) type, 10-to-15% are Lewy Bodies type, with the remaining types being of an entire spectrum of dementias including frontotemporal lobar degeneration, alcoholic dementia, pure vascular dementia.

Pure autonomic failure, also known as Bradbury-Eggleston syndrome or idiopathic orthostatic hypotension, is a form of dysautonomia that first occurs in middle age or later in life; men are affected more often than women. It is one of three diseases classified as primary autonomic failure. The symptoms concern a degenerative disease of the peripheral nervous system, symptoms include dizziness and fainting (caused by orthostatic hypotension), visual disturbances and neck pain. Chest pain, fatigue and sexual dysfunction are less common symptoms that may also occur. Symptoms are worse when standing; sometimes one may relieve symptoms by laying down. Accumulation of alpha-synuclein in autonomic nerves causes pure autonomic failure (Horacia Kaufmann et al. Neurology 2001; 56:980-981).

The term “pharmaceutically acceptable” is used herein to mean that the modified noun is appropriate for use in a pharmaceutical product.

As used herein, the term “pharmaceutically acceptable carrier” includes also any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations.

The term “treatment” refers to any process, action, application, therapy, or the like, wherein a mammal, including a human being, is subject to medical aid with the object of improving the mammal's condition, directly or indirectly. In the current invention “treatment” also refers to prevention. When a synucleinopathy is prevented it means here that the occurrence of alpha synuclein toxicity such as oxidative stress induced by alpha-synuclein or necrosis induction by alpha synuclein is suppressed as compared with the mammal not treated with an endonuclease G inhibitor of the invention. Suppression means that alpha synuclein toxicity, the endonuclease G catalyses nucleotide degradation or synucleinopathy occurs for at least 20%, 30%, 40%, 50%, 60%, 70%, 80%, 90% or even 100% less than compared with the mammal as compared with the mammal not treated with an inhibitor of endonuclease G of the invention.

The invention provides the use of a compound that inhibits the expression and/or of a endonuclease G for the manufacture of a medicament for treatment or prevention of α-synuclein toxicity.

The term ‘a compound that inhibits the expression’ refers here to gene expression and thus to the inhibition of gene transcription and/or translation of a gene transcript (mRNA) such as for example the endonuclease G gene or endonuclease G mRNA. Preferably said inhibition is at least 20%, 30%, 40%, 50%, 60%, 70%, 80%, 90% or even higher. The term ‘a compound that inhibits the activity’ refers here to the protein that is produced such as the endonuclease G protein. Said inhibition of activity leads to a diminished interaction of endonuclease G with its substrates and a diminished endonuclease G nuclease activity where under the catalyzed DNA degradation and an inhibition of the endonuclease G dependent apoptosis. Preferably said inhibition is at least 20%, 30%, 40%, 50%, 60%, 70%, 80%, 90% or even higher.

The present disclosure shows that alpha synuclein toxicity is significantly suppressed if endonuclease G is inhibited and that alpha synuclein toxicity can be suppressed by the usage of inhibitors of endonuclease G. Thus in one embodiment the present invention also relates to the usage of molecules which comprise a region that can specifically bind to endonuclease G and consequently said molecules interfere with the binding of endonuclease G to its target DNA with the interference on the endonuclease G catalyzed DNA degradation and said molecules can be used for the manufacture of a medicament for treatment of alpha synuclein toxicity and the synucleinopathies that it induces.

Thus more specifically the invention also relates to molecules that neutralize the nuclease activity of endonuclease G by interfering with its synthesis, translation, dimerization, substrate-binding and/or endonuclease G dependent pathways. By molecules it is meant peptides, peptide aptamers, tetrameric peptides, proteins, organic molecules, mutants of the DNA substrate of endonuclease G, soluble substrates of endonuclease G and any fragment or homologue thereof having the same neutralizing effect as stated above. Also, the invention the molecules comprise antagonists of endonuclease G such as anti-endonuclease G antibodies and functional fragments derived thereof, anti-sense RNA and DNA molecules and ribozymes that function to inhibit the translation of endonuclease G, all capable of interfering/or inhibiting the endonuclease G catalyzed DNA degradation or inhibiting the EndoG-dependent pathways.

By synthesis it is meant transcription of endonuclease G. Small molecules can bind on the promoter region of endonuclease G and inhibit binding of a transcription factor or said molecules can bind said transcription factor and inhibit binding to the endonuclease G-promoter.

By endonuclease G it is meant also its isoforms, which occur as a result of alternative splicing, and allelic variants thereof.

Antagonists of endonuclease G can suppress the alpha synuclein toxicity in said synucleinopathy. In a specific embodiment said synucleinopathy is Parkinson's disease, dementia with Lewy bodies, pure autonomic failure, and multiple system atrophy. With 20 “suppression” it is understood that suppression of alpha synuclein toxicity can occur for at least 20%, 30%, 30%, 50%, 60%, 70%, 80%, 90% or even 100%. More specifically the invention relates to the use of molecules (antagonists) to neutralize the activity of endonuclease G by interfering with its synthesis, translation, its activity to cleave chromatin DNA into nucleosomal fragments, its release of mitochondria or its translocation to the nucleus. By molecules it is meant peptides, tetrameric peptides, proteins, organic molecules, mutants of the endonuclease G, soluble protein or peptide ligands of the endonuclease G and any fragment or homologue thereof having the same neutralizing effect as stated above.

Also, the invention is directed to anti-endonuclease G antibodies and functional fragments derived thereof, anti-sense RNA and DNA molecules and ribozymes that function to inhibit the translation of endonuclease G, all capable of interfering/or inhibiting the endonuclease G apoptosis pathway. By synthesis it is meant transcription of endonuclease G. Small molecules can bind on the promoter region of endonuclease G and inhibit binding of a transcription factor or said molecules can bind said transcription factor and inhibit binding to the endonuclease G-promoter. By endonuclease G it is meant also its isoforms, which occur as a result of alternative splicing, and allelic variants thereof.

To inhibit the activity of the gene or the gene product of endonuclease G custom-made techniques are available directed at three distinct types of targets: DNA, RNA, and protein. For example, the gene or gene product of endonuclease G can be altered by homologous recombination, the expression of the genetic code can be inhibited at the RNA level by antisense oligonucleotides, interfering RNA (RNAi) or ribozymes, and the protein function can be altered or inhibited by antibodies or drugs.

With “inhibition of expression” to gene expression is understood the inhibition of gene transcription and/or translation of a gene transcript (mRNA) such as for example the endonuclease G gene. Preferably said inhibition is at least 20%, 30%, 40%, 50%, 60%, 70%, 80%, 90% or even higher. With “inhibiting activity” is referred to the protein that is produced such as endonuclease G or its substrates. The inhibition of activity leads to a diminished interaction (e.g., in the case of endonuclease G with its substrates and an inhibition of endoG cleaves chromatin DNA into nucleosomal fragments). Preferably said inhibition is at least 20%, 30%, 40%, 50%, 60%, 70%, 80%, 90% or even higher.

The term ‘antibody’ or ‘antibodies’ relates to an antibody characterized as being specifically directed against endonuclease G or any functional derivative thereof including Nuc1p, with said antibodies being preferably monoclonal antibodies; or an antigen-binding fragment thereof, of the F(ab′)2, F(ab) or single chain Fv type, of the single domain antibody type or any type of recombinant antibody derived thereof. These antibodies of the invention, including specific polyclonal antisera prepared against endonuclease G, or any functional derivative thereof, have no cross-reactivity to others proteins. The monoclonal antibodies of the invention can for instance be produced by any hybridoma liable to be formed according to classical methods from splenic cells of an animal, particularly of a mouse or rat immunized against endonuclease G or any functional derivative thereof, and of cells of a myeloma cell line, and to be selected by the ability of the hybridoma to produce the monoclonal antibodies recognizing endonuclease G or any functional derivative thereof which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP99/03605 or by using transgenic non-human animals capable of producing human antibodies as described in U.S. Pat. No. 5,545,806. Also fragments derived from these monoclonal antibodies such as Fab, F(ab)′2 and ssFv (“single chain variable fragment”), providing they have retained the original binding properties, form part of the present invention. Such fragments are commonly generated by, for instance, enzymatic digestion of the antibodies with papain, pepsin, other proteases. It is well known to the person skilled in the art that monoclonal antibodies, or fragments thereof, can be modified for various uses. The antibodies involved in the invention can be labeled by an appropriate label of the enzymatic, fluorescent, or radioactive type.

Small molecules, e.g., small organic molecules, and other drug candidates can be obtained, for example, from combinatorial and natural product libraries.

Random peptide libraries, such as the use of tetrameric peptide libraries such as described in W00185796, consisting of all possible combinations of amino acids attached to a solid phase support, or such as a combinatorial library of peptide aptamers, which are proteins that contain a conformationally constrained peptide region of variable sequence displayed from scaffold as described in Colas et al. (Nature 380: 548-550, 1996 and Geyer et al., Proc. Natl. Acad. Sco. USA 96: 8567-8572, 1999), may be used in the present invention.

Also transdominant-negative mutant forms of ENDOG-ligands can be used to inhibit endonuclease G dependent pathways and the ENDOF catalyses nucleotide breakdown.

Also within the scope of the invention is the use of oligoribonucleotide sequences, that 30 include anti-sense RNA and DNA molecules and ribozymes that function to inhibit the translation of endonuclease G mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of the endonuclease G nucleotide sequence, are preferred.

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of endonuclease G sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable.

Both anti-sense RNA and DNA molecules and ribozymes of the invention may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize anti-sense RNA constitutively or inducibly, depending on the promoter used, can be introduced stably into cell lines.

### LEGENDS TO THE FIGURES

FIG. 1 Age-dependent α-synuclein-mediated death is accompanied by phenotypic manifestations of apoptosis and necrosis.

(A) Survival during chronological ageing of BY4741a cells expressing human α-synuclein (wt aSyn) or the point mutant A53T under a GAL promoter or harboring the corresponding empty vector during chronological ageing. A representative ageing experiment is shown, with data representing mean±SEM of 4 independent experiments performed at the same time.

(B) Quantification of ROS accumulation using DHE-staining at day 1, 3, and 5 of α-synuclein or A53T expression in BY4741a cells. In each experiment, at least 5·10⁶ cells were evaluated. Data represent mean±SEM of 4 independent experiments.

(C) Quantification of DNA-fragmentation using TUNEL-staining at day 2 of chronological ageing of BY4741 cells expressing α-synuclein or AS3T or harboring the empty vector. In each experiment at least 30,000 cells were evaluated using flow cytometry.

(D) Quantification of AnnexinVIPI costaining, indicating phosphatidylserine externalization and membrane integrity, of BY4741a cells expressing α-synuclein or A53T for 3 days or harboring the corresponding vector control. In each experiment 30,000 cells were evaluated using flow cytometry.

FIG. 2 α-synuclein mediated death and ROS-production depends on functional mitochondria.

(A & B) Survival during chronological ageing of BY4741a wild type (A) and rho⁰ cells (B) expressing human α-synuclein (wt αSyn) or the mutant A53T (A53T) or harboring the corresponding empty vector. A representative ageing experiment is shown, with data representing mean±SEM of 4 independent experiments performed at the same time.

(C) Survival determined by clonogenicity of BY4741 wild .type (wt) and rho⁰ cells expressing α-synuclein and vector controls at indicated time points during prolonged expression on 1.5% galactose/0.5% glucose synthetic media. Data represent mean±SEM of 3 independent experiments.

(D) Quantification of ROS accumulation using DHE-staining at indicated time points of prolonged expression of α-synuclein and A53T in BY4741a wild type (wt) and rho⁰ cells. In each experiment, 30,000 cells were evaluated using flow cytometry. Data represent mean±SEM of 3 independent experiments.

(E) Quantification of AnnexinVIPI costaining, indicating phosphatidylserine externalization and membrane integrity, of BY4741a wild type (wt) and rho⁰ cells expressing α-synuclein and A53T or harboring the corresponding empty vector. In each experiment 30,000 cells were evaluated using flow cytometry.

(F) Western Blot analysis of α-synuclein or A53T expression in the background of BY4741 wild type (lanes 2 and 3) and rho⁰ (lanes 3 and 4) cells. Blot was probed with a-FLAG-antibody or a-GAPDH and the corresponding secondary antibodies.

FIG. 3 α-synuclein-mediated death of aged cells is independent of the yeast caspase YCA1, the apoptosis inducing factor AIF1 and the serine protease HtrA2/0MI (NMA111).

(A) Survival during chronological ageing of BY4741 wild type (wt) and isogenic Δyca1, Δaif1 or Δnma111 cells expressing human α-synuclein (wt aSyn) or harboring the corresponding empty vector. A representative ageing experiment is shown, with data representing mean±SEM of 4 independent experiments performed at the same time.

(B) Quantification of ROS accumulation using DHE-staining at day 1, 3, and 5 of chronological ageing of BY4741a wild type (wt) and isogenic Δyca1, Aaif1 or Anma111 cells expressing α-synuclein or harboring the corresponding empty vector. In each experiment, at least 5·10⁶ cells were evaluated. Data represent mean±SEM of 4 independent experiments.

FIG. 4 Deletion of the endonuclease G (Nuc1p) suppresses α-synuclein-mediated death during early phases of ageing.

(A) Survival during chronological ageing of BY4741a (wt) and isogenic Δnuc1 cells expressing human α-synuclein (wt αSyn) or harboring the corresponding empty vector during chronological ageing. A representative ageing experiment is shown, with data representing mean±SEM of 4 independent experiments performed at the same time.

(B) Quantification of ROS accumulation using DHE-staining at day 1, 2, 3, 4 and 5 of α-Synuclein expression in BY4741a (wt) and isogenic Δnuc1 cells. In each experiment, at least 5·10⁶ cells were evaluated. Data represent mean±SEM of 4 independent experiments.

(C) Survival determined by clonogenicity of BY4741a wild type and Δnuc1 cells expressing α-synuclein and vector controls at indicated time points during prolonged expression on 1.5% galactose/0.5% glucose synthetic media. Data represent mean±SEM of 3 independent experiments.

(D) Quantification of DNA-fragmentation using TUNEL-staining at the indicated time intervals of chronological ageing of BY4741a (wt) and isogenic Δnuc1 cells expressing α-synuclein or harboring the empty vector. In each experiment at least 30,000 cells were evaluated using flow cytometry.

(E) Quantification of AnnexinV/PI costaining, indicating phosphatidylserine externalization and membrane integrity, of BY4741a (wt) and isogenic Δnuc1 cells expressing α-Synuclein or harboring the corresponding vector control. Samples were taken at the indicated times after induction of α-synuclein. In each experiment 30,000 cells were evaluated using flow cytometry.

### EXAMPLES

### Example 1

### Materials and Methods

### Yeast Strains And Molecular Biology

Experiments were carried out in BY4741 (MATa his3Δ1 leu2Δ0 met15Δ0 ura3Δ0) and respective null mutants, obtained from Euroscarf, or in W303-1A (MATa can1-100 ade2-1 his3-11 trp1-1 ura 3-1 leu 2-3, 112). All strains were grown on SC medium containing 0.17% yeast nitrogen base (Difco), 0.5% (NH₄)2S0₄ and 30 mg/l of all amino acids (except 80 mg/l histidine and 200 mg/l leucine), 30 mg/l adenine, and 320 mg/l uracil with 2% glucose (SCD), or 2% galactose (SCG) for induction of expression of α-synuclein-FLAG constructs. For abrogation of the mitochondrial DNA, BY4741a were grown in YEPD media.

### Plasmids

Plasmids for constitutive expression of native α-synuclein under control of the TPI promoter were previously described (Zabrocki et al., 2005). To construct α-synuclein-FLAG and A53T-FLAG, the cDNAs for wild type and A53T α-synuclein were introduced into pESC-His (Stratagene) where expression is controlled by the glucose-repressible but galactose-inducible GAL1 promoter.

### Survival Plating and Test for Apoptotic Markers

Chronological ageing were performed as described (Herker et al., 2004; Madeo et al., 2002). Notably, at least three different clones were tested for the survival tests to rule out clonogenic variation of the effects. AnnexinV/PI co-staining and TUNEL staining were performed as described (Madeo et al., 1997), with modifications during TUNEL-procedure: Incubation of spheroblasts with 0.3% H₂0₂ in methanol was omitted and procedure was stopped after labeling with dUTP-FITC and analyzed by fluorescence microscopy. For evaluation of TUNEL-stained cells using flow cytometry, the staining was performed in eppendorf tubes. To determine the frequency of morphological phenotypes, either 1500 cells were manually counted or 30,000 cells were evaluated using flow cytometry and BD FACSDiva software.

For dihydroethidium staining, 5·10⁶ cells were harvested by centrifugation, resuspended in 250 μl of 2.5 μg/ml DHE in PBS and incubated in the dark for 5 min. Relative fluorescence units (RFU) were determined using a fluorescence reader (Tecan, GeniusPRO™) or positive cells were counted using flow cytometry. Same samples were analyzed by fluorescence microscopy.

### Immunoblotting

Preparation of cell extracts and immunoblotting was performed as described (Madeo et al., 2002). Blots were probed with murine monoclonal antibodies against FLAG (Sigma), murine monoclonal antibodies against GAPDH (Sigma) and the respective peroxidase-conjugated affinity-purified secondary antibody (Sigma).

### Example 2

### Death in Ageing Cultures Mediated by Heterologous Expression of Human α-Synuclein is Accompanied by Phenotypic Manifestations of Apoptosis and Necrosis.

Though many neurodegenerative disorders are tightly associated with ageing, the relationship between α-synuclein-mediated toxicity, ageing and cell death has not been fully elucidated. Therefore, we applied yeast chronological ageing, a well-established model for regulation of ageing in post-mitotic mammalian cells and to date the best studied physiological scenario of apoptosis induction in wild type yeast (Fabrizio et al., 2004; Herker et al., 2004) to further characterize age-dependent α-synuclein-mediated toxicity.

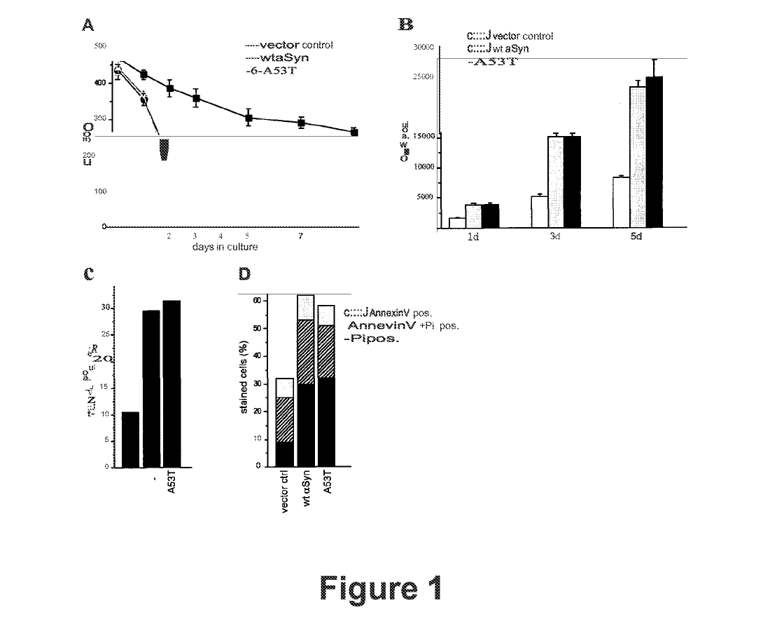

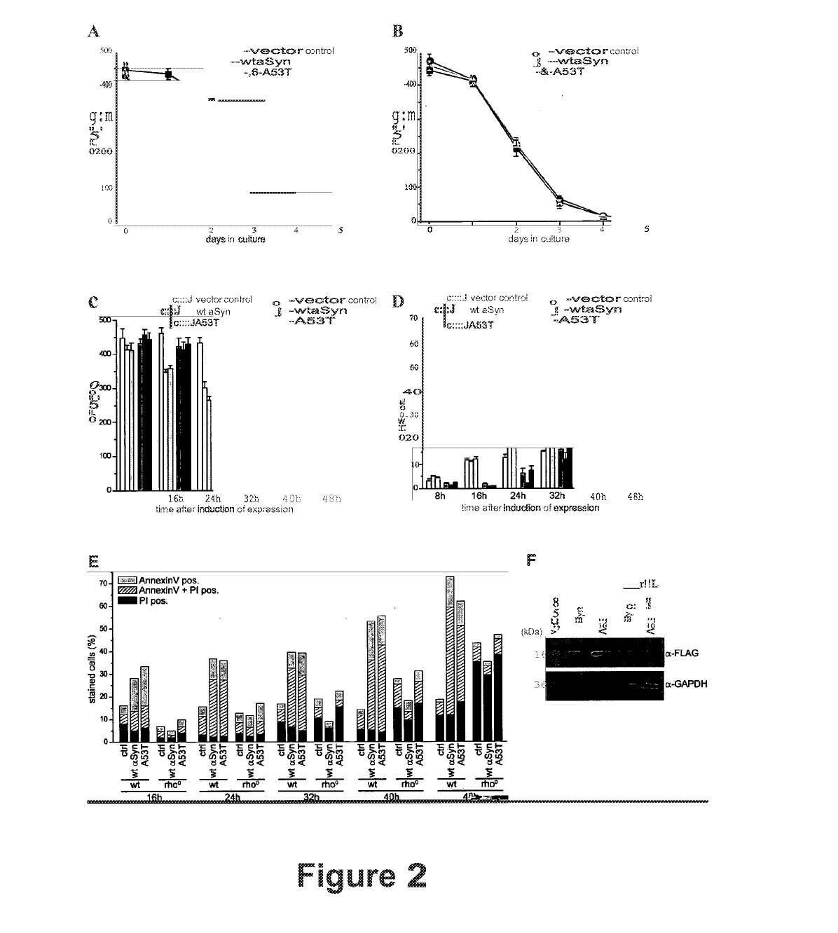

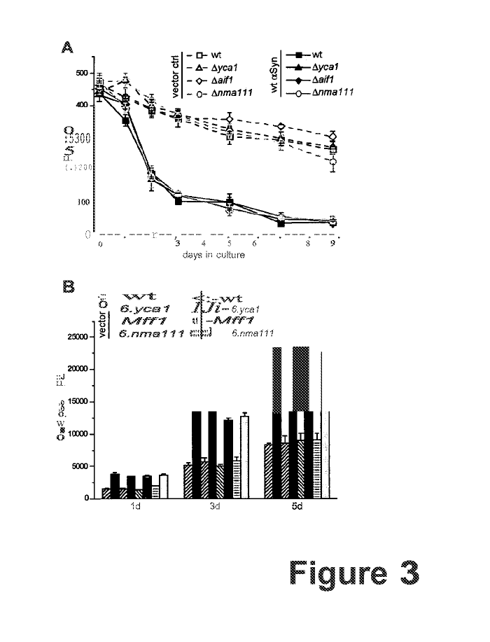

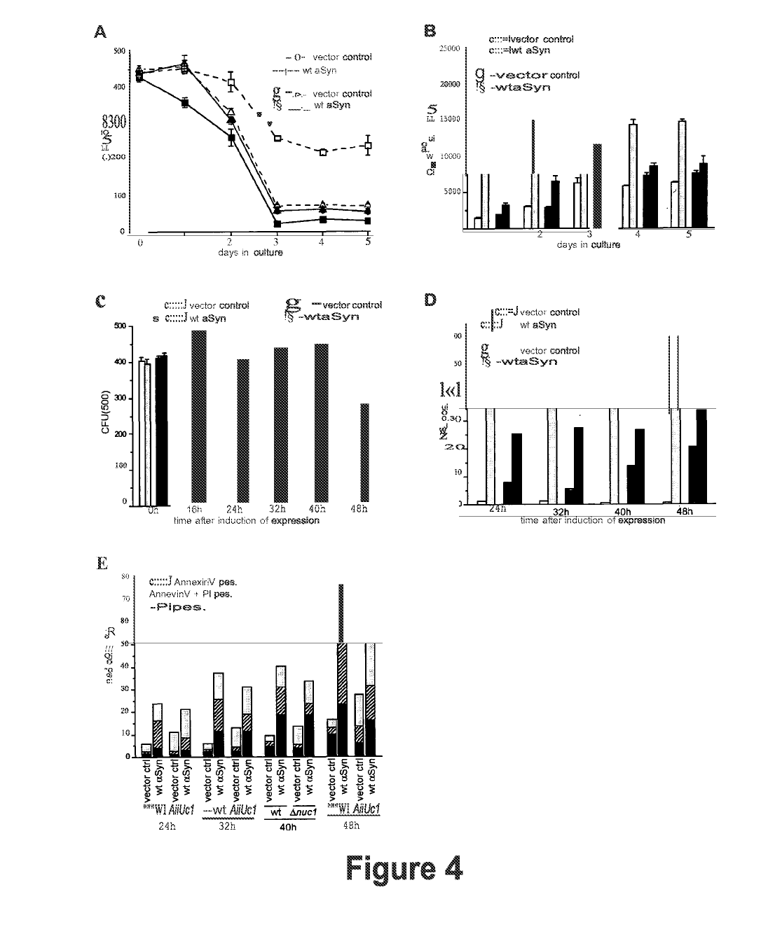

We expressed native wild type α-synuclein (wt-Syn) and a mutated variant (A53T) found in early-onset hereditary transmitted PD under the control of an inducible GAL promoter in BYa wild type yeast cells and determined survival during ageing. As shown in FIG. 1A, expression of wt-Syn led to rapid cell killing. After 4 days of expression, only ˜20% of the cells expressing the wild type protein were still able to form colonies, compared to ˜90% of the cells harboring the empty vector. The point mutation A53T shows a similar cell death as the wild type synuclein (FIG. 1A).

We next investigated whether α-synuclein-mediated death of aged yeast cells is of apoptotic nature. To quantify accumulation of reactive oxygen species (ROS), dihydroethidium (DHE)-staining was used. Automatic measurement of the relative DHE-fluorescence revealed that prolonged expression of wt-Syn (and also of A53T) leads to massive accumulation of ROS at all-time points determined (FIG. 1B). Additionally, the excessive death of synuclein expressing cells was accompanied by a large increase in apoptotic DNA-fragmentation as indicated by TUNEL-staining (FIG. 1C). Interestingly, analysis of phosphatidylserine externalization using AnnexinV and concomitant determination of membrane integrity using propidiumiodide (PI) revealed that death mediated by wt-Syn is only partially apoptotic. AnnexinVIPI costaining allows a discrimination between early apoptotic (AnnexinV pos.), late apoptotic (AnnexinV/PI pos.), and necrotic (PI pos.) cell death. Expression of synuclein enhanced the externalization of phosphatidylserine and simultaneously led to an increase in cells only positive for PI as indicative of necrosis (FIG. 1D). Interestingly, the increase of necrotic cells was most pronounced during the first 3 days of ageing while the increase in apoptotic cells continued at later time points. Similar results were obtained using the wild type yeast cells W303-1A transformed with constructs allowing expression of α-synuclein from the constitutive TPI1 promoter in (data not shown).

### Example 3

### α-Synuclein Mediated Death and ROS-Production Depends on Functional Mitochondria but is Independent of the Unfolded Protein Response (UPR).

ROS-accumulation is a prominent phenotype during ageing and apoptosis of organisms ranging from yeast to mammals. Reportedly, ROS are generated mainly from two sources: the UPR-regulated oxidative folding machinery and the mitochondria. The accumulation of misfolded proteins within the endoplasmic reticulim (ER) leads to prolonged activation unfolded protein responses (UPR), which in turn causes oxidative stress and finally cell death (Haynes et al., 2004). To test whether Synuclein-mediated death depends on the UPR-activated cell death pathway, α-synuclein was expressed in the deletion mutants of two key players of the UPR-activation, Δire1 and Δhac1. Ire1 p initiates the unfolded protein response by regulating the synthesis of the transcription factor Had p (Sidrauski and Walter, 1997; Welihinda and Kaufman, 1996; Welihinda et al., 1999). Neither deletion of IRE1 nor HAC1 affected α-synuclein-mediated toxicity (data not shown), suggesting a pathway in which UPR-signaling via Ire1p and Had p does not contribute to the loss of viability following α-synuclein expression.

As mammalian and yeast) apoptosis are under mitochondrial control (Eisenberg et al., 2007; Fannjiang et al., 2004; Ludovico et al., 2002; Wissing et al., 2004), we next investigated the impact of mitochondria and oxidative phosphorylation on death promoted by α-synuclein expression. We therefore treated cells with ethidium bromide generating cells lacking mitochondrial DNA (rho⁰ and therefore respiration capacity. Survival of BY4741 wild type and rho⁰ cells expressing wt-Syn or A53T or harboring the empty vector was determined during the first five days of chronological ageing (FIG. 2A, B). While expression of α-synuclein led to significantly increased death in the wild type, cell survival was not affected in rho⁰ cells although Western blot analysis confirmed similar expression levels (FIG. 2F). It should be noted that the lack of respiratory function via abrogation of mtDNA compromised overall survival during ageing, as these cells are no longer able to switch from fermentation to respiration during the diauxic shift. Hence, it could be argued that abrogation of mitochondrial function itself means a prodeath stimulus which cleans all putative apoptotic cells from the culture and therefore enriching the culture with survivors that are resistant against death induction by α-synuclein for trivial reasons. We therefore decided to perform a highly resolved clonogenicity analysis of wt-Syn- and A53T-mediated death during early time points of expression in wild type and rho0 cells. FIG. 2C clearly shows that wt-Syn and A53T expression led to massive cell killing in the wild type at early time points of ageing whereas death was completely inhibited in rho⁰ cells. In addition, we could further strengthen this conclusion by performing a FACS based analysis to quantify ROS-accumulation, phosphatidylserine externalization, and membrane integrity at different time points. This time course clearly demonstrates that deletion of the mtDNA not only reduced death upon α-synuclein expression, but almost completely inhibited ROS-generation (FIG. 2D) and phosphatidylserine externalization (FIG. 2E).

Thus, abrogation of mitochondrial DNA and therefore respiratory function inhibits the deadly effect of α-synuclein.

### Example 4

### α-Synuclein Mediated Death of Aged Cells is Independent of the Yeast Caspase YCA1, the Apoptosis Inducing Factor AIF1, the Serine Protease Htra2/0MI(NMA111) and Components of the Autophagic Machinery

To gain further insights into the mechanisms of α-synuclein-mediated cell killing during chronological ageing, we analyzed whether this death depends on the yeast caspase Yca1 p (Madeo et al., 2002), the apoptosis-inducing factor Aif1 p (Wissing et al., 2004), or the apoptotic serine-protease OMI (Nma111p) (Fahrenkrog et al., 2004). Furthermore, survival was monitored upon expression of wt-Syn or the point mutant A53T, which is also known to be toxic in yeast (Outeiro and Lindquist, 2003; Zabrocki et al., 2005).

FIG. 3A shows that deletion of YCA1 in the background of BY4741 had no effect on cell survival upon prolonged wt-Syn expression. Similar results were obtained with the mutant A53T (data not shown). Consistently, we could rule out an effect of YCA1 deletion on α-Synuclein-produced ROS, using a fluorescence reader to quantify DHE-detectable ROS-accumulation during chronological ageing (FIG. 38). These results were confirmed using dihydrorhodamine (DHR)-staining as another ROS-sensitive dye to detect oxidative stress (data not shown). Quantification of additional apoptotic markers via flow cytometry further confirmed that Yca1 p does not influence α-synuclein-facilitated cell killing. After 4 days of α-synuclein expression, DNA-fragmentation (TUNEL) was detectable in 27% and 23.9% of wild type BY4741 cells and in 29.5% and 31.4% of Δyca1 mutant cells transformed with wt-Syn or A53T, respectively (data not shown). AnnexinVIPI-costaining revealed that the percentage of cells showing phosphatidylserine externalization and/or membrane permeabilization upon prolonged wt-Syn or A53T expression was not altered upon YCA1 deletion (data not shown). Furthermore, neither deletion of AIF1 nor Nma111 could reduce α-synuclein-mediated cell killing (FIG. 3A) or ROS-production during chronological ageing (FIG. 3B).

Thus, our data indicate that during ageing, disruption of YCA1, AIF1, or OMI has neither an effect on α-synuclein-mediated death nor on the phenotypic changes indicative of necrosis or apoptosis.

### Example 5

### Deletion of the Endonuclease G (Nuc1p) Suppresses α-Synuclein-Mediated Death During Early Phases Of Ageing

Most recently, we identified the yeast mitochondrial endonuclease G, Nuc1, as a novel cell death regulator in yeast that induces apoptosis independently of the metacaspase Yca1p or the apoptosis inducing factor Aif1p (Buttner et al., 2007a). In order to test the involvement of Nuc1 in α-synuclein-mediated death; we expressed Synuclein in the background of a strain deleted in Nuc1p. While expression of α-synuclein led to significantly increased death in the wild type, cell survival was restored by deletion of the yeast endonuclease G (Nuc1p) during early phases of ageing (FIG. 4A). As reported previously (Buttner et al., 2007a) lack of Nuc1p compromised overall survival of the cells, but only during late phases of ageing. Consistently, ROS accumulation, a defining feature of the ageing process was drastically diminished in the same time frame by deletion of Nuc1p (FIG. 4B). To further investigate this effect, we perfoni1ed a highly resolved clonogenicity analysis of wt-Syn-mediated death during early time points of expression in wild type and Δnuc1 cells. FIG. 4C clearly shows that wt-Syn and expression led to massive cell killing in the wild type at early time points of ageing whereas death was completely inhibited in Δnuc1 cells for 40 h of ageing. In addition, we could further strengthen this conclusion by performing a FACS based analysis to quantify DNA strand breaks, phosphatidylserine externalization, and membrane integrity at different time points (FIG. 4D, E). This time course demonstrates that deletion of Nuc1p not only reduced death upon α-synuclein expression, but also diminished apoptotic markers like DNA cleavage and phosphatidylserine externalization.

Thus, abrogation of the yeast endonuclease G inhibits the deadly effect of α-synuclein.

### Example 6

### Knockdown of the Endonuclease G Suppresses α-Synuclein-Mediated Death in Human Neuroblastoma SHSY5Y Cells

To provide further biological relevance of above mentioned results, a cell culture model for PD based on human neuroblastoma SHSY5Y cells, is used (Hasegawa T. et al., Brain Research 1013: 51-59, 2004). Cells are grown in Dulbecco's modified Eagle's medium (DMEM, Gibco-BRL, Invitrogen, Belgium) supplemented with 15% fetal calf serum (International Medical, Belgium), 50 μg gentamicin solution (Gibco-BRL) and 1% non-essential amino acids (Gibco-BRL) (further referred to as DMEM-complete) at 37° C. and 5% C02 in a humidified atmosphere. Methods to enhance and monitor α-synuclein aggregation have been described previously (W02005109004; Ostrerova-Golts et al. (J. Neurosci. 20: 6048-6054, 2000 and Gerard et al).

The method of immunoblotting and Western blot analysis have been described in detail elsewhere (Ostrerova-Golts et al. J. Neurosci. 20: 6048-6054, 2000; Niikura et al. J. Cell Bioi 178: 283-296, 2007). Primary antibodies directed against α-synuclein and EndoG are purchased from Sigma-Aldrich (Bornem, Belgium) and Abeam (Cambridge, UK), respectively. Knock-down of EndoG is obtained by siRNA molecules previously described. Three sets of EndoG siRNAs have been described by Niikura et al. (J. Cell Biol. 178: 283-296, 2007) that display similar efficiencies for depletion of cells for EndoG activity: 5′-AAGAGCCGCGAGUCGUACGUG-3′ (SEQ. ID. 1), 5′-AACGCACCUGUGGAUGAGGCC-3′ (SEQ. ID 2), and 5′-CGGGCUUCGGGGCUGCUCUUU-3′ (SEQ. ID 3). In addition, Basnakian et al. (Experimental Cell Research 312: 4139-4149, 2006) carried out EndoG siRNA silencing with an siRNA duplex (sense siRNA 5′-AUGCCUGGAACAACCUGGAdTdT-3′ (SEQ. ID 4) antisense siRNA 3′-CCAGGUUGUUCCAGGCAUdTdT-5′ (SEQ. ID 5)). The siRNA molecules are purchased at Qiagen (Germantown, Md., USA). For transfection, 125000 SHSYSY cells are plated in a 24-well plate. The following day, the cells are transfected with the siRNAs using siFECTamine (IC-Vec ltd, London, UK) as transfection reagent. Per well, 0.91 μl siRNA (200 μM) is mixed with 220μ1 OptiMEM (Gibco-BRL) and 9.6 μl siFECTamine. The solution is vortexed and incubated at room temperature for 5 minutes. 1OO μl OptiMEM is applied on the cells after a washing step with OptiMEM. Next the siRNA mixture is added to the well. The cells are then incubated with this transfection medium for at least 6 hours. Subsequently, the medium is replaced with OMEM-complete. The efficiency of EndoG down regulation is measured 24 to 48 hours after transfection by western blot analysis.

For visualization of α-synuclein aggregation as well as for evaluation of the effect of EndoG on α-synuclein aggregation, 10 mM FeCl₂ and 100 μM H₂0₂ are added to the siRNA transfected and non-transfected SHSYSY cells and cells are incubated for another three days. After fixation, aggregates are visualized with thioflavin S and the percentage of aggregate positive cells is determined. As control serves cells that display normal expression of EndoG.

To confirm that α-synuclein-induced toxicity in SHSY5Y cell-based PD model is mediated via EndoG, cells with or without siRNA mediated knock-down of EndoG are compared for their level of reactive oxygen species, mitochondrial activity and apoptotic cell death parameters. Apoptotic SHSY5Y cells are quantified using an AnnexinV-FITC/PI kit and FACS flow cytometry as described previously (Lin et al. Biochem J 406: 215-221, 2007; Lee et al., Exp. Mol. Med. 39:376-384, 2007). Cells in the early stages of apoptosis are Annexin V positive; whereas, cells that are Annexin V and PI positive are in the late stages of apoptosis. The determination of ROS levels and cytosolic cytochrome c is also described by Lin et al. (Biochem J 406: 215-221, 2007). Apoptotic cells are further detected by the terminal deoxynucleotidyltransferase-mediated dUTP-biotin nick end labeling (TUNEL) assay using the In Situ Cell Death Detection kit with fluorescein (Roche Applied Science) according to the instructions provided by the manufacturer. Detailed methods for the determination of cell viability and mitochondrial membrane potential are described by Lee et al. (Exp. Mol. Med. 39:376-384, 2007).

The studies clearly demonstrate that alpha synuclein toxicity such as α-synuclein mediated cell death, a synuclein induced reactive oxygen species (ROS) in a cell requires the proapoptotic endonuclease G and that knock down of the endonuclease G apoptotic pathway attenuates or counteracts such alpha synuclein toxicity.

### Further Illustrative Embodiments

In this study, we investigated α-synuclein-mediated toxicity during chronological ageing of yeast cells in clonogenic assays and combined those with the measurement of apoptotic markers. During chronological ageing, yeast cells reach the stationary phase and are then kept in the exhausted medium. Since the cells do then not divide anymore, the ageing test represents a model for studying cell viability in a condition comparable to the post-mitotic neurons in higher eukaryotes.

In short, our studies indicate that overexpression of wt-Syn or the clinical A53T mutant dramatically reduces longevity of the yeast cells due to a marked increase in ROS and the induction of apoptosis, the latter being independent of the UPR-regulated oxidative folding machinery but strictly associated to mitochondrial functions and in particular the yeast endonuclease G, Nuc1. These data extend previous observations made in yeast showing that α-synuclein-induced toxicity is related to the ability of wt-Syn and A53T to interact with the plasma membrane and to form inclusions, in contrast to A30P (Outeiro and Lindquist, 2003; Zabrocki et al., 2005). Our data also generally agree with a recent report on the induction of ROS and apoptosis in yeast cells expressing α-synuclein, with exception of the involvement of the metacaspase, Yca1p, which was found to abolish the α-synuclein-induced ROS accumulation in peroxide treated cells (Flower et al., 2005). Notably, the involvement of Yca1p was already questioned before, as its deletion did not ameliorate the α-synuclein cytotoxicity in cells grown in the presence of Zn² or Fe³⁺ (Griffioen et al., 2006).

A critical evaluation of our data, challenges some of the current hypotheses on the mechanisms leading to degeneration of the dopamine producing neurons. As α-synuclein is rather ubiquitously expressed in brain, the selective susceptibility of neurons in the substantia nigra was attributed to oxidative damage caused by dopamine metabolites. Accordingly, α-synuclein protofibrils can form pores in vesicular membranes leading to permeabilization and the release of dopamine into the cytosol where it is metabolized and oxidized, causing increased production of free radicals and oxidative stress (Lashuel et al., 2002; Sulzer et al., 2000). Moreover, since dopamine metabolites promote and stabilize protofibril formation and oxidative stress is known to exacerbate α-synuclein toxicity, it was proposed that cells enter a upward spiral where dopamine and α-synuclein enhance each other's toxicity (Abou-Sieiman et al., 2006; Conway et al., 2001; Wood-Kaczmar et al., 2006; Xu et al., 2002). Also in yeast cells expressing α-synuclein such a vicious circle is likely to occur as previous studies revealed oxidative stress to enhance a-Synuclein toxicity and aggregation (Griffioen et al., 2006; Zabrocki et al., 2005) while this study shows that expression of the toxic wt-Syn and A53T to be sufficient to induce enhanced ROS formation. However, yeast cells do not produce dopamine and a survey of the available databases did not reveal any gene that could be associated with monoamine or catecholamine metabolism. Therefore, our study indicates that effects triggered by dopamine metabolism are not essential and that properties of α-synuclein itself, eventually in combination with other factors, can be regarded as the primary cause leading to cell death. Notably, recent studies in yeast convincingly showed that there is no strict correlation between enhanced α-synuclein aggregation and toxicity (Voiles and Lansbury, 2007).

Several studies focused on excitotoxic effects caused by defective mitochondrial energy metabolism leading to decreased ATP production and hypothesized that deregulation of the NMDA subtype glutamate receptor or malfunctioning of the ATP-sensitive potassium channels could be the underlying mechanism for selective dopaminergic degeneration in PD (Beal, 1992; Greenamyre et al., 1999; Liss and Roeper, 2001). Again those mechanisms have no direct analogue in the yeast model and therefore might be of secondary importance, though being still relevant as to establish feed-forward loops in a neuronal context triggering increased oxidative stress and accumulation of ROS. Note, however, that this does not exclude the possibility that, given its property to interact with membranes, α-synuclein may itself have a direct effect on ion homeostasis as discussed below.

One may argue that the α-synuclein-induced toxicity in yeast is merely the result of expression of the heterologous fibrillar protein of which the yeast cell wants to dispose, and that toxicity is caused by overloading the proteasome degradation systems and failure to remove endogenous misfolded, oxidized or damaged proteins. Although inhibition of the proteasome was shown to dramatically increase aggregation of wt-Syn in yeast cells (Zabrocki et al., 2005) such a scenario cannot explain the difference in toxicity observed between wild type and some α-synuclein mutants, including the clinical A30P mutant which is maintained at much higher levels (Outeiro and Lindquist, 2003; Voiles and Lansbury, 2007; Zabrocki et al., 2005). As mentioned above, there is also no strict correlation between increased aggregation and α-synuclein-mediated toxicity in yeast (Voiles and Lansbury, 2007).

In addition and despite of recent observations indicative for enhanced UPR activity in mammalian cellular models and brain of PO patients (Hoozemans et al., 2007; Smith et al., 2005; Yamamuro et al., 2006), we could not confirm UPR to be a primary cause for the loss of viability since the lack of Ire1 p and Had p did not alleviate α-synuclein-induced toxicity in yeast.

One of the proteases that was recently associated with PO is Omi/HtrA2 (Strauss et al., 2005). This serine protease is released from the innermembrane space by opening of the mitochondrial permeability transition pore (mPTP) as a consequence of depolarization of the membrane potential. Once in the cytosol Omi/HtrA2 binds inhibitor of apoptosis proteins (IAPs) thereby relieving the inhibition of caspases. The yeast ortholog of Omi/HtrA2, Nma111p, fulfills similar functions as it binds to the IAP Bir1p to prevent apoptosis induced by H₂0₂-mediated oxidative stress (Fahrenkrog et al., 2004; Walter et al., 2006). However, we found that deletion of NMA111/0MI, similar to deletion of the metacaspase Yca1 (Madeo et al., 2002) or the apoptosis inducing factor AIF (Wissing et al., 2004), did not protect yeast cells from α-synuclein-induced apoptosis, indicative that the protein is not directly involved. Instead, we identified Nuc1p, an homolog of mammalian endonuclease G (Buttner et al., 2007a), to directly execute α-synuclein-mediated cell death. To date, no links between EndoG and PO have been described but the endonuclease has been associated with degenerative diseases such as cerebral ischemia (Lee et al., 2005) and muscle atrophy (Leeuwenburgh et al., 2005). Interestingly, Nuc1 interacts in yeast cells with the adenosine nucleotide translocator Aac2p and the voltage dependent anion channel Por1p/YVDAC1, both subunits of the mPTP, and the karyopherin Kap123p, which is involved in nuclear import (Buttner et al., 2007b). These proteins all have their homologs in mammalians and at least the ortholog of MC2, ANT2, was found to be specifically upregulated in mesostriatal dopaminergic neurons, which preferentially degenerate in PD (Chung et al., 2005). In addition, another mPTP subunit, the peripheral benzodiazepine receptor homologue PBR was upregulated in Drosophila parkin mutants (Abou-Sieiman et al., 2006; Casellas et al., 2002). Finally, Kap123 has importin-β3 as closest human homolog. Importin-β proteins serve in retrograde injury signalling and their expression is rapidly induced in injured nerve cells by local axonal translation. This allows the creation of heterodimer α/β importin complexes with high affinity binding sites for nuclear localization signal, which in turn couple to the retrograde motor dynein (Hanz and Fainzilber, 2004).

In conclusion, our studies identified Nuc1, the orthologs of mammalian EndoG, as central executor of α-synuclein-induced apoptosis. So far, EndoG has not been associated to PD but it was implicated in several other degenerative disorders. Once more these studies show the potential offered by yeast models for defining novel fundamental mechanisms and factors involved in the pathogenesis of PD.

The studies clearly demonstrate that alpha synuclein toxicity such as α-synuclein mediated cell death, a synuclein induced reactive oxygen species (ROS) in a cell requires the proapoptotic endonuclease G and that the deletion of the endonuclease G or suppressing of the endonuclease G apoptotic pathway attenuates or counteracts such alpha synuclein toxicity.

Thus molecules recognizing and inhibiting EndoG can be used to counteract such α-synuclein toxicity, α-synuclein induced oxidative stress in cells and tissues and α-synuclein mediated cell death.

Such molecules for inhibiting the expression or the activity of endonuclease G or their methods of preparation are available in the art such as nucleotides, antibodies, ribozymes, tetrameric peptide which can be conjugated with domains to internalize in the cell for instance protein transduction domains. For instance such nucleotide is typically an antisense DNA or RNA, siRNA, miRNA or an RNA aptamer. Such protein transduction domains or cell penetrating peptides (CPPs) have been demonstrated to be useful for delivery of a wide range of macromolecules including peptides, proteins and antisense oligonucleotides. For instance Bryan R. Meadea and Steven F. Dowdy (Advanced Drug Delivery Reviews Volume 59, Issues 2-3, 30 Mar. 2007, Pages 134-140) demonstrate efficient exogenous siRNA delivery using peptide transduction domains/cell penetrating peptides. Methods for noncovalent complexing of CPPs with siRNA or covalent attachment of CPPs to siRNA are available in the art for successful gene delivery into cells (I. A. Ignatovich, et al. J. Bioi. Chem. 278 (2003), pp. 42625-42636, C. Rudolph, et al. J. Bioi. Chem. 278 (2003), pp. 11411-11418, S. Sandgren, et al. J. Bioi. Chem. 277 (2002), pp. 38877-38883, N. Unnamalai, et al. FEBS Lett. 566 (2004), pp. 307-310., F. Simeoni, et al. Nucleic Acids Res. 31 (2003), pp. 2717-2724, S. Sandgren, et al. J. Bioi. Chem. 277 (2002), pp. 38877-38883, A. Muratovska and M. R. Eccles, FEBS Lett. 558 (2004), pp. 63-68 and Y. L. Chiu et al. Chem. Bioi. 11 (2004), pp. 1165-1175), and T. J. Davidson et al. described highly efficient small interfering RNA delivery to primary mammalian neurons induces microRNA-like effects before mRNA degradation (J. Neurosci. 24 (2004), pp. 1004G-10046). Specific Knockdown of EndoG by siRNA and the induced reduction of EndoG expression by the delivery of siRNA plasmids constructs into Vero cells (cell lineage isolated from kidney epithelial cells extracted from African green monkey (Cercopithecus aethiops)) and the design of suitable constructs has also been also described by Ke-Jung Huang et al in PNAS Jun. 13, 2006 vol. 103 no. 24 8995-9000.

The inhibiting nucleotides of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise a inhibiting nucleotides of the invention (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, 0.3% glycine, hyaluronic acid and the like.

Pharmaceutical compositions. of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of one or more inhibiting nucleotide of the invention. A pharmaceutical composition for aerosol (inhalational) administration can comprise 0.01-20% by weight, preferably 1%-10% by weight, of one or more inhibiting nucleotide of the invention encapsulated in a liposome as described above, and propellant. A carrier can also be included as desired; e.g., lecithin for intranasal delivery.

Over the past decade, miRNAs and siRNAs have emerged as important regulators of translation and mRNA decay. The regulatory pathways mediated by these small RNAs are usually collectively referred to as RNA interference (RNAi) or RNA silencing. For instance Bahi N, et al. J Bioi Chern 2006; 281: 22943-2295 carried out silencing of EndoG by small hairpin RNA Interference (shRNAi) using lentiviral vectors. Alexei G. Basnakian (Experimental Cell Research Volume 312, Issue 20, 10 Dec. 2006, Pages 4139-4149) carried out EndoG siRNA silencing to knockdown EndoG mRNA, cells by transfection with designed siRNA duplexes (sense siRNA 5′-AUGCCUGGAACAACCUGGAdTdT-3′ (SEQ. ID 6) antisense siRNA 3′-UCCAGGUUGUUCCAGGCAUdTdT-5′ (SEQ. ID 7)) and demonstrated its efficacy by control with Non-Targeting siRNA #1 (Dharmacon, Lafayette, Colo.). Three sets of EndoG siRNAs have been described by Yohei Niikura et al. (J. Cell Bioi 178: 283-296, 2007) that display similar efficiencies for depletion of Hela cells for EndoG activity: 5′-AAGAGCCGCGAGUCGUACGUG-3′ (SEQ. ID 8), 5′-AACGCACCUGUGGAUGAGGCC-3′, and 5′-CGGGCUUCGGGGCUGCUCUUU-3′ (SEQ. ID 9). Jinming Yang et al (Clinical Cancer Research Vol. 12, 950-960, February 2006) demonstrated the knock down of EndoG protein in human cells 96 hours after three successive rounds of siRNA EndoG transfection. According to methods of: RNA Interference and delivery of small interfering RNA to mammalian cells described by Robert M. Brazas and James E. Hagstrom in Methods in Enzymology Volume’ 392, 2005, Pages 112-124 Matthew Whiteman et al employed RNA interference (siRNA) to knock down EndoG protein expression (Cellular Signalling Volume 19, Issue 4, April 2007, Pages 705-714). Jay Parrish et al carried out Caenorrhabditis elegans endoG(RNAi) to silence the EndoG homologie cps-6 (Nature Vol. 412 5 Jul. 2001).

Inhibition of BNIP3 by RNAi is known to inhibit the EndoG mediated apoptosis pathway. Such shRNA sequence that is of high-inhibition efficiency for BNIP3 have already been identified and demonstrated to inhibit EndoG. For instance Zhang, Zhengfeng et al. (Stroke: Volume 38(5) May 2007 pp 1606-1613) described 12 pairs of oligonucleotides were initially designed, synthesized, and cloned into Invitrogen pENTRIU6 vectors. The vectors were cotransfected with the BNIP3-expressing plasmid pcDNA3-haBNIP3 into HEK 293 cells. The inhibition efficiencies of BNIP3 varied from none to almost complete inhibition as determined by immunofluorescence microscopy and quantitative Western blot analysis (data not shown). One pair of oligonucleotides (N167, forward, 5′-CACCGCTTCCGTCTCTATTTATATTCAAGAGATATAAATAGAGACGGAAGC-3′ (SEQ. ID 10); backward, 5′-AAAAGCTTCCGTCTCTATTTATATCTCTTGAATATAAATAGAGACGGAAGC-3′ (SEQ. ID 11) (bold, sense and antisense strands; underlined, loop) targeting the nucleotides 167 to 188 in the BNIP3 mRNA sequence (GenBank accession number NM_{—}053420) showed the most potent inhibition. Quantification of the Western blot bands revealed that the inhibition efficiency of the N167 for hamster and rat BNIP3 expression was 98.1% as compared with the nontransfected controls. To inhibit BNIP3 expression in neurons, lentiviral vector carrying the N167 sequence were developed. Transfection of primary cortical neurons with the N167 lentiviral vector resulted in complete inhibition of BNIP3 in neurons exposed to hypoxia for 48 hours whereas no inhibition of BNIP3 was observed with a lentiviral vector carrying the (control) LacZ sequence and a vector carrying a scrambled sequence (S167) that contained the same nucleotide composition as N167. They demonstrated that inhibition of BNIP3 can delay for instance hypoxia-induced EndoG translocation by 24 hours. BNIP3 RNAi (HSS141388, HSS141389 and HSS141390) and endonuclease G RNAi (HSS141943 HSS141944 and HSS141945) for gene knock-down are available from Invitrogen.

Compounds that antagonise BNIP3 activity, compositions containing such compounds, and methods of use of the compounds have been provided for reducing or preventing α-synuclein toxicity by inhibiting BNIP3 activity and are also an object of the present invention. Also provided are methods of treatment or amelioration of one or more symptoms of diseases and disorders associated with α-synuclein toxicity and disorders associated with α-synuclein fibril formation. Such diseases and disorders include, but are not limited to, Parkinson's disease and Lewy body dementia. Other diseases and disorders include synucleinopathies, such as pure autonomic failure, and multiple system atrophy and the manufacture of medicaments for such treatment. Use of any of the described compounds for the treatment or amelioration of one or more symptoms of diseases and disorders associated with α-synuclein toxicity or α-synuclein fibril formation is also contemplated. Furthermore, use of any of the described compounds for the manufacture of a medicament for the treatment of diseases and disorders associated with α-synuclein toxicity or α-synuclein fibril formation is also contemplated. The present invention also provides a method of inhibiting or preventing α-synuclein toxicity such as oxidative stress induced by α-synuclein or necrosis induced by α-synuclein whereby a composition that comprises at least one BNIP3 inhibitor is administered to a mammal or contacted with a cell.

A first embodiment of this object is a compound having an inhibitory action on BNIP3 dependent apoptosis or a compound that inhibits the expression and/or activity of BNIP3 for use in a treatment to cure or to prevent of α-synuclein toxicity associated diseases for instance the synucleinopathies or such α-synuclein toxicity associated diseases of the group consisting of Parkinson's disease, dementia with Lewy bodies, pure autonomic failure and multiple system atrophy. Such compound having an inhibitory action on BNIP3 dependent apoptosis or inhibits the expression and/or activity of BNIP3 can be a compound selected from the group consisting of a nucleotide, an antibody, a ribozyme, and tetrameric peptide. To enhance cell entry such compound can be conjugated with a protein transduction domain. The nucleotide to inhibit the expression and/or activity of BNIP3 can be an antisense DNA or RNA, siRNA, miRNA or an RNA aptamer. Suitable reducing a synuclein activity are the monoclonal antibodies specifically directed to BNIP3 or antigen-binding fragment thereof. Such antibody or antibody fragment can be humanized.

Knocking down the alpha synuclein toxicity with a siRNA that knocks down the expression of EndoG or BNIP3 is a specific object of present invention. Methods for enhancing the in vivo intracellular delivery of therapeutic oligonucleotides such as siRNA can be linked with a linking moiety to a delivery aptamer sequence as for instance described in W02005111238. Another technology available for intracellular delivery of siRNA is the passenger strand of the siRNA to cholesterol to facilitate uptake through ubiquitously expressed cell surface LDL receptors as for instance described by Soutschek et al (Nature 432, 173-178. 2004). Other systems for cell delivery of short interfering RNA (siRNA) therapeutic, directed against an intracellular target is a formulation of such siRNA as a nanocomplex with a RNAi/Oligonucleotide nanoparticle polymer delivery system (RONDEL™ Calando). The nanocomplex comprises non-chemically modified siRNA (C05C), cyclodextrin polymers (CAL101), a stabilising agent (AD-PEG) and a targeting agent (AD-PEG-Tf) which together form nanoparticles for systemic delivery. Systems have been developed for efficient delivery of the small interfering RNA to targets in neural tissue. Several formulations are available including saline, polymer complexation and lipid or liposomal formulations for efficacious delivery of siRNAs locally to the nervous system for instance via intrathecal delivery. The simplest mode of efficient delivery is intracerebroventricular, intrathecal or intraparenchymal infusion of naked siRNA formulated in buffered isotonic saline to silence specific neuronal molecular targets in multiple regions of the central and peripheral system. For delivery of small interfering RNA (siRNA) to the spinal cord and peripheral neurons has been described by Luo M C et al. (Mol Pain. 2005 Sep. 28; 1:29). On the other hand J. F. Cryan et al. (Biochem. Soc. Trans. (2007) 35, (411-415)) described gene knockdown involving chronic infusion of siRNA (short interfering RNA) using osmotic minipumps. Polymer complexation and lipid or liposomal formulations such as polyethylene imine (PEI), IFECT, DOTAP and JetSI/DOPE also facilitate cellular uptake and reduce doses of siRNA to a level that is required for effective neuronal target silencing in vivo (Tan P H et al. Gen. Ther. 12, 59-66 (2005)).

An “Aptamer” is a single- or double-stranded nucleic acid which is capable of binding to a protein or other molecule, and thereby disturbing the protein or other molecule function. Whereas an “endonuclease G aptamer”: a single- or double-stranded nucleic acid which binds to endonuclease G and disturbs its function in particular its nuclease activity.

Aptamers are nucleic acid molecules having specific binding affinity to molecules through interactions other than classic Watson-Crick base pairing. Aptamers, like peptides generated by phage display or monoclonal antibodies (mAbs), are capable of specifically binding to selected targets and modulating the target's activity, e.g., through binding, aptamers may block their target's ability to function. Created by an in vitro selection process from pools of random sequence oligonucleotides aptamers can be been generated for over m any proteins. A typical aptamer is 10-15 kDa in size (30-45 nucleotides), binds its target with sub-nanomolar affinity, and discriminates against closely related targets (e.g. aptamers will typically not bind other proteins from the same gene family). A series of structural studies have shown that aptamers are capable of using the same types of binding interactions (e.g., hydrogen bonding, electrostatic complementarities, hydrophobic contacts, steric exclusion) that drive affinity and specificity in antibody-antigen complexes. Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example: speed and control. Aptamers are produced by an entirely in vitro process, allowing for the rapid generation of initial leads, including therapeutic leads. In vitro selection allows the specificity and affinity of the aptamer to be tightly controlled and allows the generation of leads, including leads against both toxic and non-immunogenic targets. Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC, and the immune response is generally trained not to recognize nucleic acid fragments).

A suitable method for generating an nucleotide with a particular feature from highly diverse pools of different nucleotides, RNA or DNA (dsDNA or ssDNA) molecules is with the process entitled (systematic evolution of ligands by exponential enrichment) “SELEX” of G. F. Joyce (La Jolla), J. W. Szostak (Boston), and L. Gold (Boulder). (Famulok, M. and Szostak, J. W., In Vitro Selection of Specific Ligand Binding Nucleic Acids. Angew. Chem. 1992, 104, 1001 [Angew. Chem. Int. Ed. Engl. 1992, 31, 979-988]; Famulok, M. and Szostak, J. W., Selection of Functional RNA and DNA Molecules from Randomized Sequences. Nucleic Acids and Molecular Biology, Vol 7, F. Eckstein, D. M. J. Lilley, Eds., Springer VerlagBerlin, 1993, pp. 271, Klug, S.; Famulok, M., All you wanted to know about SELEX. Mol. Bioi. Reports 1994, 20, 97-107; and Burgstaller, P. and Famulok, M. Synthetic ribozymes and the first deoxyribozyme. Angew. Chem. 1995, 107, 1303-1306 [Angew. Chem. Int. Ed. Engl. 1995, 34, 1189-119].) This can be also used for generating the specific aptamer. The SELEX process is a method for the in vitro evolution of nucleic acid molecules with highly specific binding to target molecules and is described in, e.g. U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX-identified nucleic acid ligand is a specific ligand of a given target compound or molecule. The SELEX process is based on; the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. SELEX relies as a starting point upon a large library of single stranded oligonucleotides comprising randomized sequences derived from chemical synthesis on a standard DNA synthesizer. The oligonucleotides can be modified or unmodified DNA, RNA or DNNRNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a pre-selected purpose such as, CpG motifs described further below, hybridization sites for PCR primers, promoter sequences for RNA polymerases (e.g. T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a; number of aptamers that bind to the same target. The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations. The random sequence portion of the. oligonucleotide can be of any length and can comprise ribonucleotides and/or deoxyribonucleotides and can include modified or non-natural nucleotides or nucleotide analogs. (See, e.g. U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO92/07065.) Random oligonucleotides can be synthesized from phosphodiester-linked nucleotides using solid phase oligonucleotide synthesis techniques well known in the art. (See, e.g. Froehler et al., Nucl. Acid Res. 14:5399-5467 (1986) and Froehler et al., Tet. Lett. 27:5575-5578 (1986).) Random oligonucleotides can also be synthesized using solution phase methods such as triester synthesis methods. (See, e.g. Sood et al., Nucl. Acid Res. 4:2557 (1977) and Hirose et al., Tet. Lett., 28:2449 (1978).) Typical syntheses carried out on automated DNA synthesis equipment yield 1014-1016 individual molecules, a number sufficient for most SELEX experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise sketches of nonrandom or partially random sequences. Partially random sequences can be created by adding the four nucleotides in different molar ratios at each addition step.

The starting library of oligonucleotides may be either RNA or DNA. In those; instances where an RNA library is to be used as the starting library it is typically generated by transcribing a DNA library in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 420 candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 1014 different nucleic acid species but may be used to sample as many as about 1018 different nucleic acid species. Generally, nucleic acid aptamer; molecules are selected in a 5 to 20 cycle procedure. In one embodiment, heterogeneity is introduced only in the initial selection stages and does not occur throughout the replicating process. In one embodiment of SELEX, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands. In many cases, it is not necessarily desirable to perform the iterative steps of SELEX until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX process prior to completion, it is possible to determine the sequence of a number of members of the nucleic acid ligand solution family. A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX method has been modified to achieve a number of specific objectives. For example, U.S. Pat. No. 5,707,796 describes the use of SELEX in conjunction with gel electrophoresis to select nucleic acid molecules with specific structural characteristics, such as bent DNA. U.S. Pat. No. 5,763,177 describes SELEX based methods for selecting nucleic acid ligands containing photo reactive groups capable of binding and/or photo-crosslinking to and/or photo-inactivating a target molecule. U.S. Pat. No. 5,567,588 and U.S. Pat. No. 5,861,254 describe SELEX based methods which achieve highly efficient partitioning between oligonucleotides having high and low affinity for a target molecule. U.S. Pat. No. 5,496,938 describes methods for obtaining improved nucleic acid ligands after the SELEX process has been performed. U.S. Pat. No. 5,705,337 describes methods for covalently linking a ligand to its target. SELEX can also be used to obtain nucleic acid ligands that bind to more than one site on the target molecule, and to obtain nucleic acid ligands that include non-nucleic acid species that bind to specific sites on the target. SELEX provides means for isolating and identifying nucleic acid ligands which bind to any envisionable target, including large and small biomolecules such as nucleic acid-binding proteins and proteins not known to; bind nucleic acids as part of their biological function as well as cofactors and other small molecules. For example, U.S. Pat. No. 5,580,737 discloses nucleic acid sequences identified through SELEX which are capable of binding with high affinity to caffeine and the closely related analog, theophylline.

Counter-SELEX is a method for improving the specificity of nucleic acid ligands to a target molecule by eliminating nucleic acid ligand sequences with cross-reactivity to one or more non-target molecules. Counter-SELEX is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX, cycles of selection and amplification are repeated as necessary until a desired goal is achieved. One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described, e.g. in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified; pyrimidines, and U.S. Pat. No. 5,580,737 which describes highly specific nucleic acid ligands containing one or more nucleotides modified with 2′-amino (2′—NH2), 2′-fluoro (2′ F), and/or 2′-O-methyl (2′-OMe) substituents.

Modifications of the nucleic acid ligands contemplated in this invention include, but are 35 not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5 iodo-uracil; backbone modifications, phosphorothioate or alkyl phosphate modifications, methylations, and unusual base-pairing combinations such as the isobases, isocytidine, and isoguanosine. Modifications can also include 3′ and 5′ modifications such as capping. In one embodiment, oligonucleotides are provided in which the P(O)0 group is replaced by P(O)S (“thioate”), P(S)S (“dithioate”), P(O)NR2 (“amidate”), P(O)R, P(O) 0 R′, CO or CH2 (“formacetal”) or 3′-amine (—NH—CH2-CH2-), wherein each R or R′ is independently H or substituted or unsubstituted alkyl. Linkage groups can be attached to adjacent nucleotides through an-0-, -N-, or -S-linkage. Not all linkages in the oligonucleotide are required to be identical. As used herein, the term phosphorothioate encompasses one or more non-bridging oxygen atoms in a phosphodiester bond replaced by one or more sulfur atom.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group.

Methods of synthesis of 2′-modified sugars are described, e.g. in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX process modifications or post-SELEX process modifications (modification of previously identified unmodified ligands) or may be made by incorporation into the SELEX process.

Pre-SELEX process modifications or those made by incorporation into the SELEX process yield nucleic acid ligands with both specificity for their SELEX target and improved stability, e.g., in vivo stability. Post-SELEX process modifications made to nucleic acid ligands may result in improved stability, e.g., in vivo stability without adversely affecting the binding capacity of the nucleic acid ligand.

The SELEX method encompasses combining selected oligonucleotides with other selected oligonucleotides and non-oligonucleotide functional units as described in U.S. Pat. No. 5,637,459 and U.S. Pat. No. 5,683,867. The SELEX method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P. an 11 amino acid peptide, were identified.

The aptamers with specificity and binding affinity to the target(s) of the present invention are typically selected by the SELEX process as described herein. As part of the SELEX process, the sequences selected to bind to the target are then optionally minimized to determine the minimal sequence having the desired binding affinity. The selected sequences and/or the minimized sequences are optionally optimized by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo. 2′ Modified SELEX. In order for an aptamer to be suitable for use as a therapeutic, it is preferably inexpensive to synthesize, safe and stable it’ vivo. Wild-type RNA and DNA aptamers are typically not stable in vivo because of their susceptibility to degradation by nucleuses.

Resistance to nuclease degradation can be greatly increased by the incorporation of modifying groups at the 2′-position. Fluoro and amino groups have been successfully incorporated into oligonucleotide pools from which aptamers have been subsequently selected. However, these modifications greatly increase the cost of synthesis of the resultant aptamer, and may introduce safety concerns in some cases because of the possibility that the modified nucleotides could be recycled into host DNA by degradation of the modified oligonucleotides and subsequent use of the nucleotides as substrates for DNA synthesis. Aptamers that contain 2′-0-methyl (“2′-0Me”) nucleotides, as provided herein, overcome many of these drawbacks. Oligonucleotides containing 2′-0Me nucleotides are nuclease-resistant and inexpensive to synthesize. Although 2′-0Me nucleotides are ubiquitous in biological systems, natural polymerases do not accept 2′-0Me NTPs as substrates under physiological conditions, thus there are no safety concerns over the recycling of 2′-0Me nucleotides into host DNA.

The SELEX method used to generate 2′-modified aptamers is described, e.g. in U.S. Provisional Patent Application Ser. No. 60/430, 761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, and U.S. patent application Ser. No. 10/873,856, filed Jun. 21, 2004, entitled “Method for in vitro Selection of 2′-0-methyl Substituted Nucleic Acids”, each of which is herein incorporated by reference in its entirety.

### Enhanced α-Synuclein Toxicity

As specific protein binding protein or peptide ligands, antibodies can be custom-made for virtually any given protein, due to the clonal selection and maturation function of the immune system. Antibodies raised against specific proteins have made possible many technological advances in the field of molecular biology, including modern immunochemistry (Harlow and Lane, Antibodies: A Laboratory Manual, Cold Spring Harbor Laboratory Press, Cold Spring Harbor, N.Y. (1988)). The term ‘antibody’ or ‘antibodies’ relates to an antibody characterized as being specifically directed against endonuclease G or any functional derivative thereof, with said antibodies being preferably monoclonal antibodies; or an antigen-binding fragment thereof, of the F(ab′)2, F(ab) or single chain Fv type, a single domain antibody or any type of recombinant antibody derived thereof. Preferably these antibodies, including specific polyclonal antisera prepared against endonuclease G or any functional derivative thereof, have no cross-reactivity to others proteins. Preparation of Anti-endonuclease G antibodies by immunization with a 12-amino acid peptide (AELPPVPGGPRG (SEQ. ID. 12)) located at amino acid 49 to amino acid 60 of human endonuclease G (12-mer peptide) with the peptide synthesis and antibody preparation, Immunoaffinity Purification of Endonuclease G has for instance been described by Ke-Jung Huang et al in J. Bioi. Chem., Vol. 277, Issue 23, 21071-21079, Jun. 7, 2002.

Monoclonal antibodies can for instance be produced by any hybridoma liable to be formed according to classical methods from splenic cells of an animal, particularly of a mouse or rat immunized against endonuclease G or any functional derivative thereof, and of cells of a myeloma cell line, and to be selected by the ability of the hybridoma to produce the monoclonal antibodies recognizing endonuclease G or any functional derivative thereof which have been initially used for the immunization of the animals.

Another embodiment is the use of monoclonal antibody against endonuclease G. A preferred method to produce anti-endonuclease G is for instance by priming rats, for instance Lewis rats (Harlan Sprague-Dawley Inc., Indianapolis, Ind.) with a subcutaneous injection of an antigen comprising a murine endonuclease G fragment. Emulsified in suitable adjuvant, for instance complete Freund's adjuvant (Sigma). Rats have to receive booster intraperitoneal injections, preferably 4 such booster injections at 2-3-wk intervals with 100 mg of endonuclease G. Rats showing the highest titter of blocking antibody, for instance in a blocking assays, should consequently be boosted intravenously with such endonuclease G antigen preferably with a dose of about 50 mg. About five days later the splenocytes can be harvested and fused to mouse myeloma cells, preferably the P3-X63-Ag8.653 cells. Generation of hybridomas and subcloning is performed according to the current standard protocols available to the men skilled in the art. Hybridomas secreting anti-endonuclease G can for instance be selected for binding to soluble endonuclease G in ELISA The anti-endonuclease G can then be selected for inhibition of endonuclease G/substrate binding as described below. The binding kinetics of anti-endonuclease G can be measured using a Biacore biosensor (Pharmacia iosensor). Anti-endonuclease G can then be produced by culture of hybridoma cells in a suitable medium for instance serum-free medium and the Anti-endonuclease G can be purified from conditioned media for instance by a multistep chromatography process. Assessment for purity is generally done by SDS-PAGE and. Immunoreactivity in ELISA with an endonuclease G substrate. A negative control rat IgG can be used for comparison. Protein concentration of antibodies are usually determined using the BCA method. The efficiency of such anti-endonuclease G to block binding of endonuclease G protein or peptide ligands to their substrate can be measured by a substrate/endonuclease G blocking assays in plates coated with the peptide (GTX29647 GeneTex Inc) which used for blocking the activity of anit-EndoG antibody. After sequential incubation with EndoG-alkalinephosphatase (AP), preincubated with various concentrations of anti-EndoG, and calorigenic substrate, it is possible to measured binding by microtiter plate reading at 405 nm. EndoG-alkaline phosphatase (AP) is obtainable by fusing EndoG to human secretory alkaline phosphatase.

Several anti-endonuclease G antibodies are in the art and are available to the public. They are for the anti-endonuclease G antibody, which is available from ProSci Inc. (rabbit polyclonal #3035 EndoG Monoclonal Antibody, EndoG Monoclonal Antibody No. PM-4583, EndoG Monoclonal Antibody No. PM-4577, EndoG Monoclonal Antibody No. PM-4579)), and EndoG Monoclonal Antibody (Catalog No. PM-4581).

Expression and Purification of Recombinant EndoG can be carried out as follows: Full length human EndoG cDNA with an additional six histidine residues appended to its C-terminus, cloned into pFastBacl (Life Technologies, Inc.), is transformed into DH10Bac cells (Life Technologies, Inc.) and the recombinant viral DNA is purified according to the Bacto-Bac baculovirus expression procedure. The purified bacmids are used to transfect Sf21 insect cells using CellFECTIN reagent (Life Technologies, Inc.) and transfected cells are grown in IPL41 medium with 10% fetal calf serum, 2.6 g/liter tryptose phosphate, 4 g/liter yeastolate, and 0.1% Pluronic F-68 plus penicillin (100 units/ml), streptomycin (100 μg/ml), and fungizone (0.25 g/ml). Forty ml of the amplified viral, stock is used to infect 1 liter of cells at 2×106 cells/ml. The infected cells are harvested 2 days later, resuspended and homogenized in 5 vol of buffer T (20 mM Tris-HCl, pH 8.0, 50 mM NaCl, 1 mM 13-mercaptoethanol, and 0.1 mM PMSF) with 0.5% Nonidet P40 (NP-40). This and all subsequent operations are conducted at 4° C. The cell homogenate is centrifugated at 10,000×g for 30 min and the supernatant is loaded onto a 3 ml nickel affinity column. The column is washed with 30 ml of buffer T with 0.5% NP-40, 30 ml of buffer T, followed by 200 ml of buffer T plus 1 M NaCl. The column is washed once more with buffer T and proteins are eluted with buffer T plus 250 mM imidazole. The eluted proteins are loaded onto a Superdex 200 column (Amersham Pharmacia Biotech) and eluted with buffer A (20 mM Hepes-KOH, pH 7.0, 10 mM KCl, 1.5 mM MgCl2, 1 mM NaEDTA, 1 mM NaEGTA, 1 mM dithiothreitol, and 0.1 mM PMSF). The peak fractions are loaded onto a Mono S column (Amersham Pharmacia Biotech) and eluted with a 20 ml linear gradient from 0 mM to 300 mM NaCl in buffer A. The peak of EndoG nuclease activity, eluting at approximately 80 mM NaCl, is stored at −20° C. in 50% glycerol. Protein purity is assessed by SDS-15% polyacrylamide gel electrophoresis.

A preferred embodiment for preparing monoclonal antibodies against human

EndoG is for instance as follows: A recombinant human EndoG fusion protein, consisting of the amino acids encoded by EndoG or a fragment thereof is coupled to Glutathione S-transferase (GST) and expressed in Escherichia coli and purified by affinity chromatography on immobilized glutathione (Amersham Biosciences). Recombinant human EndoG (ALX-201-244-C020 Produced in E. coli. fused to a His-tag at the C-terminus and with purity 90% (SDS-PAGE) is obtainable from Alexis Biochemicals. Recombinant human endonuclease G is mixed with an equal amount of an adjuvant, and an obtained mixture is than subcutaneously administrated to Balb/c male mice (8 weeks old upon the start of immunization) in an amount corresponding to an amount of endonuclease G of 100 μg per 1 mouse (priming immunization). After about 21 days, immunization can be performed by subcutaneous administration in the same manner as described above (booster immunization). After 19 days or 30 days from the booster, the mice can administrated through their tail veins with 200 μl of a preparation obtained by diluting human endonuclease G with PBS (phosphate-buffered physiological saline) to have a concentration of 250 μg/ml (final immunization). Spleens have than to be excised from the mice after about 3 days from the final immunization, and they have to be separate into single cells. Subsequently, the spleen cells should be washed with a proper medium, e.g. DMEM medium. On the other hand, suitable mouse myeloma cells (e.g. Sp2/0-Ag14) have to be collected in the logarithmic growth phase, and to be washed with a proper medium, e.g. DMEM medium. The spleen cells and the mouse myeloma cells have to be sufficiently mixed in a plastic tube in a ratio of numbers of the cells of 10:1, followed by addition of 50% (w/v) polyethylene glycol (PEG e.g. of Boehringer Mannheim, average molecular weight: 4000) to perform cell fusion at 37° C. for 7 minutes. After removal of the supernatant solution (by means of centrifugation), the residue is added with HAT medium (DMEM medium containing 10% fetal bovine serum added with hypoxanthine, aminopterin, and thymidine). The residue has to be suspended so that a concentration of the spleen cells of about 5×106 cells/ml is obtained. This cell suspension can then be dispensed and poured into 96-well plastic plates so that one well contains about 100 μl of the suspension, followed by cultivation at 37° C. in 5% carbon dioxide. HAT medium has to be supplemented; for instance in an amount of 50 μl/well on 2nd and 5th days. After that, half volume of the medium can be exchanged every 3 or 4 days in conformity with proliferation of hybridomas.

Screening and Cloning of Hybridomas: Hybridomas, which produce the monoclonal antibody of the present invention, have to be screened for. This has to be done by using, as an index, the inhibitory activity of the monoclonal antibody on the physiological activity possessed by endonuclease G. Hybridomas, which produced monoclonal antibodies exhibiting reactivity with endonuclease G's have then to be selected from the selected clones. The obtained hybridomas have then to be transferred to a suitable medium for instance HT medium which is the same as HAT medium except that aminopterin is removed from HAT medium, and cultured further. Cloning can be performed twice in accordance with the limiting dilution method by which stable hybridomas are obtainable.

Production and Purification of Monoclonal Antibodies: 2.6,10,14-Tetramethylpentadecane (e.g. Pristane of Sigma, 0.5 ml) can be intraperitoneally injected into Balb/c female mice (6 to 8 weeks old from the birth). After 10 to 20 days, cells of clones can be (1×106 to 107 cells) suspended in PBS and intraperitoneally inoculated into the mice. After 7 to 10 days, the mice can be sacrificed and subjected to an abdominal operation, from which produced ascitic fluid can be collected. The ascitic fluid can be centrifuged to remove insoluble matters, and a supernatant was recovered and stored at −20° C. until purification. Consequently, IgG can be purified from the ascitic fluid supernatant described above by using Hi-Trap Protein-A antibody purification kit (available from Pharmacia, Roosendaal, Netherlands). Namely, the ascitic fluid (2 ml) can be added with Solution A (1.5 M glycine, 3 M NaCl, pH 8.9, 8 ml), and filtrated with a filter for filtration having a pore size of 45 μm (Millipore). After that, an obtained filtrate can applied to a column (column volume: 1 ml) charged with Protein Sepharose HP (produced by Pharmacia) sufficiently equilibrated with Solution A, and the column has be washed with Solution A in an amount of 10-fold column volume. Subsequently, an IgG fraction can be eluted with Solution B (0.1 M glycine, pH 2.8) in an amount of 10-fold column volume. The eluted IgG fraction can be dialyzed against PBS. The monoclonal antibodies can be determined for their IgG subclasses by using the purified antibodies obtained in the foregoing, by means of a commercially available subclass-determining kit (trade name: Mono Ab-ID EIA Kit A, produced by Zymed). This method is based on the ELISA method.

Antibody fragments can nowadays be isolated from naive phage display libraries without immunisation, by-passing hybridoma technology (Winter et al. (1994) Annu. Rev. Immunol. 12, 433). The method of Pini, A., et al J. Biol. Chem. (1998) 273, 21769-21776 is used to design a phage display libraries of human antibodies with subnanomolar affinity against endonuclease G from such library, monoclonal antibody fragments against a virtually infinite number of different antigens can be produced. The antibodies can be expressed in bacteria (typical yields: 1-50 mg/litre in shaker flasks) and affinity-purified on Protein A Sepharose. They can be used for practically all standard antibody-based assays (western blotting, ELISA, immunohistochemistry, immunoprecipitation, etc.). Very limited equipment (normally available in Biochemistry or Molecular Biology laboratories) is required. Typically, 1-2 weeks of (limited amount of) work are necessary to produce antibodies against a purified antigen, by a normally skilled scientist.

The Inhibitory Activities of Monoclonal Antibodies can be tested for complete inhibition of the nuclease activity of endonuclease G. This can for instance measured in an immunofunctional ELISA in which 96-well plates are coated with 100 μl of 1 μg/ml of rmFlt-1/Fc chimera overnight at room temperature in PBS. After blocking for 1 hour with 1% BSA in PBS, 100 μl of a mixture of 70 μl of hybridoma medium pre-incubated with 70 μl of recombinant mENDOG-2 at 10 ng/ml for 2 hours at room temperature is then applied to the plate. A standard of rmENDOG-2 ranging 25 from 20 ng/ml to 156 pg/ml can be included (diluted in PBS-Tween.BSA-EDTA). Plates can then be incubated 1 hour at 37° C. and 1 hour at room temperature, washed 5 times with PBS-Tween and 100 pi of biotinylated goat anti-endonuclease G at 200 ng/ml can be applied for 2 hours at room temperature. After washing 5 times with PBS-Tween, 100 μl of avidin-HRP conjugate (Vectastorin ABC kit) can be applied for 1 hour at room temperature. After washing 5 times with PBS-Tween, the plate can be developed with 90 μl of o-phenylene diamine in citrate phosphate buffer pH 5.0 for 30 minutes and measured at 490 nm.

The present invention also provides inhibiting antibody protein or peptide ligands, which are able to bind to endonuclease G. More preferably, such a ligand should be able to recognize a specific epitope located on endonuclease G. For instance, the present invention relates to protein or peptide ligands of the above mentioned type, being derived from a monoclonal antibody produced by on purpose immunization in animals. The present invention also provides an antigen-binding Fab fragment, or a homologue derivative of such fragment, which may be obtained by proteolytic digestion of the said monoclonal antibody by papain, using methods well known in the art. In order to reduce the immunogenicity of the anti-endonuclease G monoclonal antibody, the present invention also includes the construction of a chimeric antibody, preferentially as a single-chain variable domain, which combines the variable region of the mouse antibody with a human antibody constant region-a so-called humanized monoclonal antibody.

The monoclonal antibodies produced in animals may be humanized, for instance by associating the binding complementarily determining region (“CDR”) from the non-human monoclonal antibody with human framework regions-in particular the constant C region of human gene-such as disclosed by Jones et al. in Nature (1986) 321:522 or Riechmann in Nature (1988) 332:323, or otherwise hybridized.

The monoclonal antibodies may be humanized versions of the mouse monoclonal antibodies made by means of recombinant DNA technology, departing from the mouse and/or human genomic DNA sequences coding for H and L chains or from cDNA clones coding for H and L chains. Alternatively monoclonal antibodies may be human monoclonal antibodies. Such human monoclonal antibodies are prepared, for instance, by means of human peripheral blood lymphocytes (PBL) repopulating of severe combined immune deficiency. (SCID) mice as described in PCT/EP 99/03605 or by using transgenic non-human animals capable of producing human antibodies as described in U.S. Pat. No. 5,545,806. Also fragments derived from these monoclonal antibodies such as Fab, F(ab)′2 and ssFv (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies can also be labelled by an appropriate label of the enzymatic, fluorescent, or radioactive type.

A preferred embodiment for preparing of F(ab′)2 or monovalent Fab fragments is for instance as follows: In order to prepare F(ab′)2 fragments, the monoclonal antibody can be dialysed overnight against a 0.1 mol/L citrate buffer (pH 3.5). The antibody (200 parts) are then digested by incubation with pepsin (1 part) available from Sigma (Saint-Louis, Mo.) for 1 hour at 37° C. Digestion is consequently stopped by adding 1 volume of a 1 M Tris HCl buffer (pH 9) to 10 volumes of antibody. Monovalent Fab fragments can prepared by papain digestion as follows: a 1 volume of a 1M phosphate buffer (pH 7.3) is added to 10 volumes of the monoclonal antibody, then 1 volume papain (Sigma) is added to 25 volumes of the phosphate buffer containing monoclonal antibody, 10 mmol/l L-Cysteine HCl (Sigma) and 15 mmol/L ethylene diaminetetra-acetic acid (hereinafter referred to as EDTA). After incubation for 3 hours at 37° C., digestion is stopped by adding a final concentration of 30 mmol/l freshly prepared iodoacetamide solution (Sigma), keeping the mixture in the dark at room temperature for 30 minutes. Both F(ab′)2 and Fab fragments can further be purified from contaminating intact IgG and Fe fragments using protein-A-Sepharose. The purified fragments can finally dialysed against phosphate-buffered saline (herein after referred as PBS). Purity of the fragments can be determined by sodium dodecylsulphate polyacrylamide gel electrophoresis and the protein concentration can be measured using the bicinchonicic acid Protein Assay Reagent A (Pierce, Rockford, Ill.).

Present invention provides also a method for treating alpha synuclein toxicity in an individual, said method comprising administering an antagonist of endonuclease G to that individual in an amount effective to treat said a synucleinopathy, wherein said antagonist is an anti-endonuclease G antibody or a functionally active fragment thereof. Particular suitable are the antibody fragments such as Fabs, the single-chain variable fragment miniantibodies (scFv) or the single domain antibodies. The construction of antibody fragment constructs, such as Fabs (Better, M., Chang, C. P., Robinson, R. R., and Horwitz, A. H. 1988. Science 240: 1041-1043.), Fvs (Skerra, A. and Pluckthun., A. 1988. Science 240: 1038-1041), scFvs (Bird, R. E., et al. 1988 Science 242: 423-426), dsFvs (Reiter, Y., et al. 1996. Nat. Biotechnol. 14: 1239-1245), and even single-domain VHs (Ward, E. S., et al. 1989. Nature 341: 544-546; Cai, X. and Garen, A. 1996. Proc. Natl. Acad. Sci. 93:628<H5285) and Single-domain antibody fragments (Mireille Dumoulin et al. Protein Science (2002), 11:500-515), which can be expressed in E. coli, yeast (Horwitz, A. H., Chang, C. P., Better, M., Hellstrom, K. E., and Robinson, R. R. 1988. Secretion of functional antibody and Fab fragment from yeast cells. Proc. Natl. Acad. Sci. 85: 8678-8682) or myeloma cells (Riechmann, L., Foote, J., and Winter, G. 1988). Expression of an antibody Fv fragment in myeloma cells. J. Mol. Bioi. 203: 825-828) are well documented. Antibodies in scFv format consist of a single polypeptide chain, comprising an antibody heavy chain variable domain (VH) linked by a flexible polypeptide linker to a light chain variable domain (VL). Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one aspect of the invention, a single domain antibody as used herein is a naturally occurring single domain antibody known as heavy chain antibody devoid of light chains. Such single domain antibodies are disclosed in WO 9404678 for example. For clarity reasons, this variable domain derived from a heavy chain antibody naturally devoid of light chain is known herein as a VHH to distinguish it from the conventional VH of four chain immunoglobulins. Such a VHH molecule can be derived from antibodies raised in Camelidae species, for example in camel, dromedary, alpaca and guanaco. Other species besides Camelidae may produce heavy chain antibodies naturally devoid of light chain; such VHHs are within the scope of the invention. VHHs, according to the present invention, and as known to the skilled addressee are heavy chain variable domains derived from immunoglobulins naturally devoid of light chains such as those derived from Camelids as described in W09404678 (and referred to hereinafter as VHH domains or nanobodies). VHH molecules are about 1O× smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognized by antibodies generated in vitro through the use of antibody libraries or via immunization of mammals other than Camelids (WO 9749805).

A protein capable of specifically interacting with an EndoG as described in this invention can be any antigen recognition protein, preferably a monovalent (i.e. has a single antigen-recognition site) single domain protein. The protein is preferably small, i.e., consisting of less than 240 amino acids, preferably consisting of 60 to 200 amino acids, more preferably consisting of 80 to 180 amino acids, more preferably consisting of 100 to 140 amino acids, and most preferably consisting of 110 to 135 amino acids.

A protein capable of specifically interacting with EndoG can be an antibody. Preferably the antibody is selected from the group of a camelid heavy chain monomer, camelid VHH antibody fragment, marine or human single domain antibody fragments affybody, camelized ScFv, or any functional fragment thereof.

Also disclosed are chimeric, humanized and/or deimmunized versions of the above mentioned monovalent single domain proteins molecule capable of specifically interacting with EndoG. Chimeric antibodies are produced by recombinant processes well known in the art, and have an animal variable region and a human constant region. Humanized antibodies correspond more closely to the sequence of human antibodies than do chimeric antibodies. In a humanized antibody, only the complementarily determining regions (CDRs), which are responsible for antigen binding and specificity, are non-human derived and have an amino acid sequence corresponding to the non-human antibody, and substantially all of the remaining portions of the molecule (except, in some cases, small portions of the framework regions within the variable region) are human derived and have an amino acid sequence corresponding to a human antibody. See L. Riechmann et al., Nature; 332: 323-327 1988; U.S. Pat. No. 5,225,539 (Medical Research Council); U.S. Pat. Nos. 5,585,089; 5,693, 761; 5,693, 762 (Protein Design Labs, Inc.).

Deimmunized antibodies are antibodies in which the antibody sequence is screened for potential EndoG-binding and/or T-cell epitope encoding amino acid sequences, followed by the introduction of amino acid substitutions to minimize the number of such potential MHC-binding and/or T-cell epitope encoding amino acid sequences. This method is described in detail in W09852976 (Biovation Ltd).

In a preferred embodiment of the invention, the single-domain protein capable of specifically binding to an MHC-peptide complex is a camelid VHH antibody fragment.

Camelidae (camels, dromedaries and llamas) as well as some sharks have unusual antibodies without light chains, termed heavy chain antibodies (Hamers-Casterman C. et al., 1993 Nature 363:446-468). These antibodies are highly stable antibodies that exist as a dimer of two heavy chains that lack the CH1 domain. However, they can also exist as single chain antibodies or as VH antibody fragments (VHH). Importantly, the CDR3 regions of the antibodies are much longer than the CDR3 regions of conventional antibodies, resulting in large protruding loops (Desmyter A. et al., 1996 Nat. Struct. Biol. 3:803-811). Camelid VHH antibody fragments are the smallest fragment of naturally occurring single-domain antibodies that have evolved to be fully functional in the absence of a light chain. Because these molecules have evolved in nature to be fully functional having a high affinity they can be discovered relatively easy. In addition, being a single-domain protein they have a unique loop structure by which they can bind into enzyme active sites and receptor clefts that make them extremely suited for application in inhibition of the active site of enzymes (Lanwereys M. et al., 1998 EMBO J. 17:3512-3520; WO 97/49805). Camelid antibodies have a high degree of format flexibility and can be easily be linked to other moieties using recombinant methods. The camelid VHH antibody fragments have a high natural similarity with human antibodies and can be humanized if needed and no immunogenicity has been observed in relevant animal studies. Finally, as camelid VHH antibody fragments are small proteins, it is relatively easy to produce large amounts of these proteins. The method to generate and produce camelid VHH antibody fragments are described, for example, in W097/49805 and in Ghahroudi M. A et al., 1997 FEBS Lett. 414:521-526., the contents of which are incorporated herein by reference.

Random peptide libraries, such as tetrameric peptide libraries further described herein, consisting of all possible combinations of amino acids attached to a solid phase support may be used to identify peptides that are able to bind to the ligand binding site of a given receptor or other functional domains of a receptor such as kinase domains (Lam K S et al., 1991, Nature 354, 82). The screening of peptide libraries may have therapeutic value in the discovery of pharmaceutical agents that act to inhibit the biological activity of enzymes through their interactions with the given receptor. Identification of molecules that are able to bind to the endonuclease G may be accomplished by screening a peptide library with recombinant soluble endonuclease G. The peptides can be conjugating with cationic protein transduction domains (PTDs)/cell penetrating peptides (CPPs) or short basic peptides derived mainly from transcription factor motifs, such as Int of Drosophila Antennapedia or the TAT peptide (of HIV-1). This embodiment also includes the screening and use of so-called peptide aptamer libraries. Peptide aptamers are proteins that contain a conformationally constrained peptide region of variable sequence displayed from a scaffold such as Escherichia coli thioredoxin (TrxA). The aptamers can be selected based on an interaction trap two-hybrid system that detects specific protein interactions disrupted by the aptamer as described by Colas et al. (Nature 380: 548-550, 1996) and Geyer et al. (Proc. Natl. Acad. Sci. USA 96: 8567-8572, 1999) or yeast two-hybrid as described by Emma Warbrick Ways & Means Yeast two hybrid mapping Structure 1997, Vol5 No 1. In mammalian cells (Cohen et al., Proc Natl. Acad. Sci. USA, 95, 14272-14277, 1998) and in Drosophila melanogaster (Kolonin et al. Proc Natl. Acad. Sci. USA, 95: 14266-14271) such peptide aptamers have been shown to function as dominant reverse genetic agents.

In another preferred embodiment of the invention, the single domain protein capable of specifically interacting with an EndoG is an anticalin. Anticalins are single domain antigen recognition molecules that are derived from natural lipocalins (Beste G. et al., 1999 Proc Nati Acad Sci USA 96:1898-1903; Korndorferi. P. et al., 2003 Proteins 53:121-129). The method to generate and use anticalins is described in detail in WO99/16873 and WO03/029471 (Pieris Proteolab AG the contents of which are incorporated herein by reference).

A custom technology to deliver such molecules which recognize EndoG for instance the antibody fragment intracellular is by protein transduction delivery. Therefor the antibody fragment is conjugating with cationic protein transduction domains (PTDs)/cell penetrating peptides (CPPs) or short basic peptides derived mainly from transcription factor motifs, such as Int of Drosophila Antennapedia or the TAT peptide (of HIV-1) for instance a scFv anti-ENDOG-Int(+) fusion protein (Avignolo C. et al. The Faseb Journal Vol. 22 Apr. 2008).

Generally, the present protein or peptide ligands against EndoG or BENIP3 such as the antibody derived compounds or the tetrameric peptides will be utilized in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The protein or peptide ligands of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with the protein or peptide ligands of the present invention.

The route of administration of pharmaceutical compositions according to the invention may be any of those commonly known to those of ordinary skill in the art. For therapy, including without limitation immunotherapy, the protein or peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The protein or peptide ligands of the invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present protein or peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of protein or peptide ligarid per kilogram of body weight, with doses of 0.05 to 2.0 mg/kg/dose being more commonly used. For prophylactic applications, compositions containing the present protein or peptide ligands or cocktails thereof may also be administered in similar or slightly lower dosages.

A composition containing a protein or peptide ligand according to the present invention may be utilized in prophylactic and therapeutic settings to aid in the alteration, inactivation, killing or removal of a select target cell population in a mammal.

A method for treating alpha synuclein toxicity in an individual, said method comprising administering an antagonist of endonuclease G to that individual in an amount effective to treat said a synucleinopathy, wherein said antagonist is a peptide or a functionally active fragment thereof. The mechanism of these peptides to enter cells is mainly macropinocytosis, a specialized form of fluid phase endocytosis. PTD transducible antibodies that target endonuclease G can be used to inhibit the alpha synuclein toxicity.

An object of the present invention is to provide a medicament for the treatment of synucleinopathies in higher mammals exhibiting a proteosomal dysfunction and oxidative stress in tissues cells such a neural tissues or cells in such mammals due to increased alpha synuclein toxicity. Another object of the invention is to provide pharmaceutical compositions useful in achieving the foregoing object.

It is shown that α-synuclein toxicity and increased α-synuclein mediated cell death is the of results increased endonuclease G catalyzed DNA degradation and that this alpha-synuclein toxicity can be attenuated by intervening in this endonuclease G apoptotic pathway.

Thus the present invention also demonstrates a method for preventing or treating α-synuclein toxicity in a subject, particularly mammalians, including human by inhibiting, preferably locoregional inhibiting the endonuclease activity of endonuclease G the release of endonuclease G from mitochondria or the translocation of endonuclease G to the nucleus.

Another embodiment is a method for preventing or inhibiting α-synuclein toxicity in a subject, particularly mammalians, including human by inhibiting the endonuclease G apoptosis pathway. Moreover the present invention shows that an endonuclease G antagonists can be used for the manufacture of a medicament for treatment of synucleinopathies such as for example Parkinson's and more specifically for the treatment of conditions where there is an

RNA has distinct advantages over small organic molecules when considering its use to inactivate protein function in vivo. An RNA encoding sequence can be linked to a promoter and this artificial gene introduced into cells or organisms. Depending on the regulatory sequence included, this provides a unique way of constructing a time and/or tissue specific suppresser gene. Such RNA expressing genes are usually smaller than protein-coding genes and can be inserted easily into gene therapy vectors. Unlike a foreign or altered protein, RNA is less likely to evoke an immune response. Antisense molecules and ribozymes have been developed as “code blockers” to inactivate gene function, with their promise of rational drug design and exquisite specificity (Altman, “RNase P in Research and Therapy,” Bio/Technology 13:327-329 administration will depend on the individual. Generally, the medicament is administered so that the protein, polypeptide, peptide of the present invention is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used and includes continuous subcutaneous delivery via an osmotic minipump. If so, the medicament may be infused at a dose between 5 and 20 μg/kg/minute, more preferably between 7 and 15 μg/kg/minute.

In another embodiment antibodies or functional fragments thereof can be used for the manufacture of a medicament for the treatment of the above-mentioned disorders. Non-limiting examples are the commercially available goat polyclonal antibody from R&D Pharmaceuticals, Abingdon, UK or the chicken polyclonal antibody (Gassmann et al., 1990, Faseb J. 4, 2528). Preferentially said antibodies are humanized (Rader et al., 2000, J. Bioi. Chem. 275, 13668) and more preferentially human antibodies are used as a medicament.

Another aspect of administration for treatment is the use of gene therapy to deliver the above mentioned anti-sense gene or functional parts of the endonuclease G gene or a ribozymes directed against the endonuclease G mRNA or a functional part thereof. Gene therapy means the treatment by the delivery of therapeutic nucleic acids to patient's cells. This is extensively reviewed in Lever and Goodfellow 1995; Br. Med. Bull., 51, 1-242; Culver 1995; Ledley, F. D. 1995. Hum. Gene Ther. 6, 1129. To achieve gene therapy there must be a method of delivering genes to the patient's cells and additional methods to ensure the effective production of any therapeutic genes. There are two general approaches to achieve gene delivery; these are non-viral delivery and virus-mediated gene delivery.

In another embodiment endonuclease G promoter polymorphisms can be used to identify individuals having a predisposition to acquire α-synuclein toxicity associated diseases. Indeed, it can be expected that promoter polymorphisms can give rise to much higher or much lower levels of EndoG. Consequently, higher levels of endonuclease G can lead to a predisposition to acquire an α-synuclein toxicity associated diseases such as synucleinopathy while much lower levels of endonuclease G can lead to a protection to acquire α-synuclein toxicity associated disease.

Present invention has now demonstrated that a pharmaceutical composition, which comprises an effect amount of a endonuclease G inhibitor or agonist and a pharmaceutically effective carrier can be used to decrease α-synuclein toxicity associated diseases and/or for blocking or preventing synucleinopathy in a subject. Such pharmaceutical composition can be to manufacture a medicament to treat a subject having a synucleinopathy or at risk of synucleinopathy formation. Such synucleinopathy can be a disorder of the group consisting of Parkinson's disease, dementia with Lewy bodies, pure autonomic failure and multiple system atrophy

It will be understood by those skilled in the art that any mode of administration, vehicle or carrier conventionally employed and which is inert with respect to the active agent may be utilised for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

The therapeutically effective amount of active agent to be included in the pharmaceutical composition of the invention depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg.

The invention provides thus compositions and methods useful for inhibiting, suppressing or ameliorating a synucleinopathy in mammals, including humans. The invention applies to human and veterinary applications. The inventive composition and method have been shown to be especially effective in preventing synucleinopathyformation. A new class of pharmaceutical compositions and methods of treatment and prevention of alpha synuclein toxicity related injury and disease is provided.

A preferred embodiment of present invention is thus the use of antagonists of endonuclease G for the manufacture of a medicament to treat synucleinopathy, this treatment of disorders of synucleinopathy is a suppression of alpha synuclein toxicity, preferably this synucleinopathy is a disorder of the group consisting of Parkinson's disease, dementia with Lewy bodies, pure autonomic failure and multiple system atrophy. The antagonist inhibiting or suppressing the activity of endonuclease G may be selected from the group consisting of antibodies, peptides, tetrameric peptides, small molecules, anti-sense nucleic acids and ribozymes.

Regarding the method for blocking or preventing synucleinopathy in a subject, this invention provides that the subject may be a human. The human may be a patient. The subject may also include other mammals; examples include dogs, cats, horses, rodents, or pigs, rabbits, among others.

The following examples more fully illustrate preferred features of the invention, but are not intended to limit the invention in any way. All of the starting materials and reagents disclose below are known to those skilled in the art, and are available commercially or can be prepared using well-known techniques. ADDITIONAL REFERENCES TO THIS APPLICATION
Abou-Sieiman, P. M., M. M. Muqit, and N. W. Wood. 2006. Expanding insights of mitochondrial dysfunction in Parkinson's disease. Nat Rev Neurosci. 7:207-19.
Beal, M. F. 1992. Does impairment of energy metabolism result in excitotoxic neuronal death in neurodegenerative illnesses? Ann Neural. 31:119-30.
Buttner, S., D. Carmona-Gutierrez, I. Vitale, M. Castedo, D. Ruli, T. Eisenberg, G. Kraemer, and F. Madeo. 2007a. Depletion of endonuclease G selectively kills polyploid cells. Cell Cycle. 6:1072-6.
Buttner, S., T. Eisenberg, D. Carmona-Gutierrez, D. Ruli, H. Knauer, C. Ruckenstuhl, C.
Sigrist, S. Wissing, M. Kollroser, K. U. Frohlich, S. Sigrist, and F. Madeo. 2007b. endonuclease G regulates budding yeast life and death. Mol Cell. 25:233-46.
Casellas, P., S. Galiegue, and A. S. Basile. 2002. Peripheral benzodiazepine receptors and mitochondrial function. Neurochem Int. 40:475-86.
Chandra, S., G. Gallardo, R. Fernandez-Chacon, O. M. Schluter, and T. C. Sudhof. 2005.
Alpha-synuclein cooperates with CSPalpha in preventing neurodegeneration. Cell. 123:383-96.
Chung, C. Y., H. Sea, K. C. Sonntag, A. Brooks, L. Lin, and 0. Isacson. 2005. Cell type-specific gene expression of midbrain dopaminergic neurons reveals molecules involved in their vulnerability and protection. Hum Mol Genet. 14:1709-25.
Conway, K. A., J. C. Rochet, R. M. Bieganski, and P. T. Lansbury, Jr. 2001. Kinetic stabilization of the alpha-synuclein protofibril by a dopamine-alpha-synuclein adduct. Science. 294:1346-9.
Dixon, C., N. Mathias, R. M. Zweig, D. A. Davis, and D. S. Gross. 2005. Alpha-synuclein targets the plasma membrane via the secretory pathway and induces toxicity in yeast. Genetics. 170:47-59.
Eisenberg, T., S. Buttner, G. Kraemer, and F. Madeo. 2007. The mitochondrial pathway in yeast apoptosis. Apoptosis. 12:1011-23.
Fabrizio, P., L. Battistella, R. Vardavas, C. Gattazzo, L. L. Liou, A. Diaspro, J. W. Dossen, E. B. Gralla, and V. D. Longo. 2004. Superoxide is a mediator of an altruistic aging program in Saccharomyces cerevisiae. J Cell Bioi. 166:1055-67.
Fahrenkrog, B., U. Sauder, and U. Aebi. 2004. The S. cerevisiae HtrA-like protein a111p is a nuclear serine protease that mediates yeast apoptosis. J Cell Sci. 117:115-26.
Fannjiang, Y., W. C. Cheng, S. J. Lee, B. Qi, J. Pevsner, J. M. McCaffery, R. B. Hill, G. Basanez, and J. M. Hardwick. 2004. Mitochondrial fission proteins regulate programmed cell death in yeast. Genes Dev. 18:2785-97.
Flower, T. R., L S. Chesnokova, C. A. Froelich, C. Dixon, and S. N. Witt. 2005. Heat shock prevents alpha-synuclein-induced apoptosis in a yeast model of Parkinson's disease. J Mol Bioi. 351:1081-100.
Gosal, D., O. A. Ross, and M. Toft. 2006. Parkinson's disease: the genetics of a heterogeneous disorder. Eur J Neural. 13:616-27.
Gourlay, C. W., L N. Carpp, P. Timpson, S. J. Winder, and K. R. Ayscough. 2004. A role for the actin cytoskeleton in cell death and aging in yeast. J Cell Bioi. 164:803-9.
Greenamyre, J. T., G. MacKenzie, T. I. Peng, and S. E. Stephans. 1999. Mitochondrial dysfunction in Parkinson's disease. Biochem Soc Symp. 66:85-97.
Griffioen, G., H. Duhamel, N. VanDamme, K. Pellens, P. Zabrocki, C. Pannecouque, F. van Leuven, J. Winderickx, and S. Wera. 2006. A yeast-based model of alpha-synucleinopathy identifies compounds with therapeutic potential. Biochim Biophys Acta. 1762:312-8.
Hanz, S., and M. Fainzilber. 2004. Integration of retrograde axonal and nuclear transport mechanisms in neurons: implications for therapeutics. Neuroscientist. 10:404-8.
Hardy, J., H. Cai, M. R. Cookson, K. Gwinn-Hardy, and A. Singleton. 2006. Genetics of Parkinson's disease and parkinsonism. Ann Neural. 60:389-98.
Haynes, C. M., E. A. Titus, and A. A. Cooper. 2004. Degradation of misfolded proteins prevents ER-derived oxidative stress and cell death. Mol Cell. 15:767-76.
Herker, E., H. Jungwirth, K. A. Lehmann, C. Maldener, K. U. Frohlich, S. Wissing, S. Buttner, M. Fehr, S. Sigrist, and F. Madeo. 2004. Chronological aging leads to apoptosis in yeast. J Cell Bioi. 164:501-7.
Hoozemans, J. J., E. S. van Haastert, P. Eikelenboom, R. A. de Vos, J. M. Rozemuller, and W. Scheper. 2007. Activation of the unfolded protein response in Parkinson's disease. Biochem Biophys Res Commun. 354:707-11.
Ikeda, S., and K. Ozaki. 1997. Action of mitochondrial endonuclease G on DNA damaged by L-ascorbic acid, peplomycin, and cis-diaminedichloroplatinum (II). Biochem Biophys Res Commun. 235:291-4.
Ikeda, S., T. Tanaka, H. Hasegawa, and K. Ozaki. 1996. Identification of a 55-KDA endonuclease in rat liver mitochondria with nucleolytic properties similar to endonuclease G. Biochem Mol Bioi Int. 38:1049-57.
Larsen, K. E., Y. Schmitz, M. D. Troyer, E. Mosharov, P. Dietrich, A. Z. Quazi, M. Savalle,
V. Nemani, F. A. Chaudhry, R. H. Edwards, L Stefanis, and D. Sulzer. 2006. Alpha-synuclein overexpression in PC12 and chromaffin cells impairs catecholamine release by interfering with a late step in exocytosis. J Neurosci. 26:11915-22.
Lashuel, H. A., B. M. Petre, J. Wall, M. Simon, R. J. Nowak, T. Walz, and P. T. Lansbury, Jr. 2002. Alpha-synuclein, especially the Parkinson's disease-associated mutants, forms pore-like annular and tubular protofibrils. J Mol Bioi. 322:1089-102.
Laun, P., A. Pichova, F. Madeo, J. Fuchs, A. Ellinger, S. Kohlwein, I. Dawes, K. U. Frohlich, and M. Breitenbach. 2001. Aged mother cells of Saccharomyces cerevisiae show markers of oxidative stress and apoptosis. Mol Microbial. 39:1166-73.
Lee, B. I., D. J. Lee, K. J. Cho, and G. W. Kim. 2005. Early nuclear translocation of endonuclease G and subsequent DNA fragmentation after transient focal cerebral ischemia in mice. Neurosci Lett. 386:23-7.
Leeuwenburgh, C., C. M. Gurley, B. A. Strotman, and E. E. Dupont-Versteegden. 2005. Age-related differences in apoptosis with disuse atrophy in soleus muscle. Am J Physiol Regullntegr Camp Physiol. 288:R1288-96.
Liss, B., and J. Roeper. 2001. ATP-sensitive potassium channels in dopaminergic neurons: transducers of mitochondrial dysfunction. News Physiol Sci. 16:214-:7.
Longo, V. D. 1999. Mutations in signal transduction proteins increase stress resistance and longevity in yeast, nematodes, fruit flies, and mammalian neuronal cells. Neurobiol Aging. 20:479-86.
Ludovico, P., F. Rodrigues, A. Almeida, M. T. Silva, A. Barrientos, and M. Code-Real. 2002. Cytochrome c release and mitochondria involvement in programmed cell death induced by acetic acid in Saccharomyces cerevisiae. Mol Bioi Cell. 13:2598: 606.
Ludovico, P., M. J. Sousa, M. T. Silva, C. Leao, and M. Code-Real. 2001. Saccharomyces cerevisiae commits to a programmed cell death process in response to acetic acid. Microbiology. 147:2409-15.
Madeo, F., E. Frohlich, and K. U. Frohlich. 1997. A yeast mutant showing diagnostic markers of early and late apoptosis. J Cell Bioi. 139:729-34.
Madeo, F., E. Frohlich, M. Ligr, M. Grey, S. J. Sigrist, D. H. Wolf, and K. U. Frohlich. 1999. Oxygen stress: a regulator of apoptosis in yeast. J Cell Bioi. 145:757-67.
Madeo, F., E. Herker, C. Maldener, S. Wissing, S. Lachelt, M. Herlan, M. Fehr, K. Lauber, S. J. Sigrist, S. Wesselborg, and K. U. Frohlich. 2002. A caspase-related protease regulates apoptosis in yeast. Mol Cell. 9:911-7.
Moore, D. J., A. B. West, V. L. Dawson, and T. M. Dawson. 2005. Molecular pathophysiology of Parkinson's disease. Annu Rev Neurosci. 28:57-87. Outeiro, T. F., and S. Lindquist. 2003. Yeast cells provide insight into alpha-synuclein biology and pathobiology. Science. 302:1772′-5.
Sidhu, A., C. Wersinger, and P. Vernier. 2004. Does alpha-synuclein modulate dopaminergic synaptic content and tone at the synapse? Faseb J. 18:637-47.
Sidrauski, C., and P. Walter. 1997. The transmembrane kinase Ire1p is a site-specific endonuclease that initiates mRNA splicing in the unfolded protein response. Cell. 90:1031-9.
Smith, W. W., H. Jiang, Z. Pei, Y. Tanaka, H. Morita, A. Sawa, V. L. Dawson, T. M. Dawson, and C. A. Ross. 2005. Endoplasmic reticulum stress and mitochondrial cell death pathways mediate A53T mutant alpha-synuclein-induced toxicity. Hum Mol Genet. 14:3801-11.
Strauss, K. M., L. M. Martins, H. Plun-Favreau, F. P. Marx, S. Kautzmann, D. Berg, T. Gasser, Z. Wszolek, T. Muller, A. Bornemann, H. Walburg, J. Downward, 0.
Riess, J. B. Schulz, and R. Kruger. 2005. Loss of function mutations in the gene encoding Omi/HtrA2 in Parkinson's disease. Hum Mol Genet. 14:2099-111.
Sulzer, D., J. Bogulaysky, K. E. Larsen, G. Behr, E. Karatekin, M. H. Kleinman, N. Turro, D. Krantz, R. H. Edwards, L. A. Greene, and L. Zecca. 2000. Neuromelanin biosynthesis is driven by excess cytosolic catecholamines not accumulated by synaptic vesicles. Proc Nat/Acad Sci US A. 97:11869-
Tofaris, G. K., and M. G. Spillantini. 2005. Alpha-synuclein dysfunction in Lewy body diseases. Mov Disord. 20 Suppi12:S37-44.
Voiles, M J., and P. T. Lansbury, Jr. 2007. Relationships between the sequence of alpha-synuclein and its membrane affinity, fibrillization propensity, and yeast toxicity. J 40 Mol Biol. 366:1510-22.
Walter, D., S. Wissing, F. Madeo, and B. Fahrenkrog. 2006. The inhibitor-of-apoptosis protein Bir1p protects against apoptosis in S. cerevisiae and is a substrate for the yeast homologue of Omi/HtrA2. J Cell Sci. 119:1843-51.
Welihinda, A. A., and R. J. Kaufman. 1996. The unfolded protein response pathway in Saccharomyces cerevisiae. Oligomerization and trans-phosphorylation of Ire1 p (Ern1p) are required for kinase activation. J Bioi Chem. 271:18181-7.
Welihinda, A. A., W. Tirasophon, and R. J. Kaufman. 1999. The cellular response to protein misfolding in the endoplasmic reticulum. Gene Expr. 7:293-300.
Wissing, S., P. Ludovico, E. Herker, S. Buttner, S. M. Engelhardt, T. Decker, A. Link, A. Proksch, F. Rodrigues, M. Corte-Real, K. U. Frohlich, J. Manns, C. Cande, S. J. Sigrist, G. Kraemer, and F. Madeo. 2004. An AIF orthologue regulates apoptosis in yeast. J Cell Bioi. 166:969-74.
Wood-Kaczmar, A., S. Gandhi, and N. W. Wood. 2006. Understanding the molecular causes of Parkinson's disease. Trends Mo/Med. 12:521-8.
Xu, J., S. Y. Kao, F. J. Lee, W. Song, L. W. Jin, and B. A. Yankner. 2002. Dopamine-dependent neurotoxicity of alpha-synuclein: a mechanism for selective neurodegeneration in Parkinson disease. Nat Med. 8:600-6.
Yamamuro, A., Y. Yoshioka, K. Ogita, and S. Maeda. 2006. Involvement of endoplasmic reticulum stress on the cell death induced by 6-hydroxydopamine in human neuroblastoma SH-SY5Y cells. Neurochem Res. 31:657-64.
Zabrocki, P., K. Pellens, T. Vanhelmont, T. Vandebroek, G. Griffioen, S. Wera, F. Van Leuven, and J. Winderickx. 2005. Characterization of alpha-synuclein aggregation and synergistic toxicity with protein tau in yeast. Febs J. 272:1386-400.

1. A method of treating a patient diagnosed with an α-synuclein toxicity associated disease comprising administering a pharmaceutically effective amount of a compound having a therapeutic action selected from the group consisting of an inhibitory action on endonuclease G dependent apoptosis, an inhibitory action on the expression of endonuclease G, and an inhibitory action on the activity of endonuclease G.

2. The method of claim 1, whereby the compound is selected from the list consisting of a nucleotide, an antibody, a ribozyme, a tetrameric peptide, a peptide aptamer, and a mutant endonuclease G protein.

3. The method of claim 1, wherein said compound is conjugated with a protein transduction domain.

4. The method according to claim 1, wherein the α-synuclein toxicity associated disease is selected from the group consisting of Parkinson's disease, dementia with Lewy bodies, pure autonomic failure, and multiple system atrophy.

5. The method according to claim 1, wherein the α-synuclein toxicity associated diseases is Parkinson's disease.

6. The method according to claim 1, wherein the inhibitory action inhibits one of either the expression or activity of endonuclease G.

7. A method of treating a patient diagnosed with an α-synuclein toxicity associated disease comprising administering a pharmaceutically effective amount of a nucleotide compound having a therapeutic action selected from the group consisting of an inhibitory action on endonuclease G dependent apoptosis, an inhibitory action on the expression of endonuclease G, and an inhibitory action on the activity of endonuclease G.

8. The method of claim 7, whereby the nucleotide is an antisense DNA or RNA, siRNA, miRNA, and an RNA or DNA aptamer.

9. A method of treating a patient diagnosed with an α-synuclein toxicity associated disease comprising administering a pharmaceutically effective amount of a antibody compound having a therapeutic action selected from the group consisting of an inhibitory action on endonuclease G dependent apoptosis, an inhibitory action on the expression of endonuclease G, and an inhibitory action on the activity of endonuclease G.

10. The method according to claim 9, wherein said antibody is one of either a monoclonal antibody or an antibody fragment specifically directed to one of either endonuclease G or antigen-binding fragment thereof.

11. The method according to claim 11, wherein said one of either the antibody or antibody fragment is humanized.

12. A method of treating a patient diagnosed with a synucleinopathy comprising administering a pharmaceutically effective amount of a compound having a therapeutic action selected from the group consisting of an inhibitory action on endonuclease G dependent apoptosis, an inhibitory action on the expression of endonuclease G, and an inhibitory action on the activity of endonuclease G.

13. The method according to claim 12, wherein the synucleinopathy is selected from the group consisting of Parkinson's disease, dementia with Lewy bodies, pure autonomic failure, and multiple system atrophy.

14. The method according to claim 12, wherein the synucleinopathy is Parkinson's disease.

15. The method of claim 12, whereby the compound is selected from the list consisting of a nucleotide, an antibody, a ribozyme, a tetrameric peptide, a peptide aptamer, and a mutant endonuclease G protein.

16. The method of claim 15, whereby the nucleotide is an antisense DNA or RNA, siRNA, miRNA, and an RNA or DNA aptamer.

17. The method according to claim 15, wherein said antibody is one of either a monoclonal antibody or an antibody fragment specifically directed to one of either endonuclease G or antigen-binding fragment thereof.

18. The method according to claim 17, wherein said one of either the antibody or antibody fragment is humanized.

19. The method of claim 12, wherein said compound is conjugated with a protein transduction domain.

20. The method according to claim 12, wherein the inhibitory action inhibits one of either the expression or activity of endonuclease G.
